SEQUENCE LISTING (92 sequences)

SEQ ID NO: 1 - RNA, 72 nt, Artificial Sequence
  miR451
  misc_feature (17)..(57): n is a, c, g, or u
cuugggaauggcaaggnnnnnnnnnnnnnnnnnnnnnnnnnnnnnnnnnnnnnnnnncuugcuauacccaga

SEQ ID NO: 2 - RNA, 250 nt, Artificial Sequence
  miR144 and miR451
  misc_feature (195)..(235): n is a, c, g, or u
uggggcccuggcugggauuucaucauauacuguaaguuugcgaugagacacuacaguauagaugauguacuaguccgggcacccccagcucuggagccugacaaggaggacaggagagaugcugcaagcccaagaagcucucugcucagccugucacaaccuacugacugccagggcacuugggaauggcaaggnnnnnnnnnnnnnnnnnnnnnnnnnnnnnnnnnnnnnnnnncuugcuauacccaga

SEQ ID NO: 3 - DNA, 22 bp, Artificial Sequence
  target sequence
aaggactttcaaaggccaagga

SEQ ID NO: 4 - DNA, 22 bp, Artificial Sequence
  target sequence
tggctgagaagaccaaagagca

SEQ ID NO: 5 - DNA, 22 bp, Artificial Sequence
  target sequence
agagcaagtgacaaatgttgga

SEQ ID NO: 6 - DNA, 22 bp, Artificial Sequence
  target sequence
tggctgctgctgagaaaaccaa

SEQ ID NO: 7 - DNA, 22 bp, Artificial Sequence
  target sequence
aggaaagacaaaagagggtgtt

SEQ ID NO: 8 - DNA, 22 bp, Artificial Sequence
  target sequence
tcaaaaaggaccagttgggcaa

SEQ ID NO: 9 - DNA, 22 bp, Artificial Sequence
  target sequence
agactacgaacctgaagcctaa

SEQ ID NO: 10 - RNA, 22 nt, Artificial Sequence
  miSNCA_02 guide
uccuuggccuuugaaaguccuu

SEQ ID NO: 11 - RNA, 22 nt, Artificial Sequence
  miSNCA_05 Guide
ugcucuuuggucuucucagcca

SEQ ID NO: 12 - RNA, 22 nt, Artificial Sequence
  miSNCA_07 guide
uccaacauuugucacuugcucu

SEQ ID NO: 13 - RNA, 22 nt, Artificial Sequence
  miSNCA_12 guide
uugguuuucucagcagcagcca

SEQ ID NO: 14 - RNA, 22 nt, Artificial Sequence
  miSNCA_13 guide
aacacccucuuuugucuuuccu

SEQ ID NO: 15 - RNA, 22 nt, Artificial Sequence
  miSNCA_15 guide
uugcccaacugguccuuuuuga

SEQ ID NO: 16 - RNA, 22 nt, Artificial Sequence
  miSNCA_16 guide
uuaggcuucagguucguagucu

SEQ ID NO: 17 - RNA, 72 nt, Artificial Sequence
  miSNCA_02 in pri-miRNA
cuugggaauggcaagguccuuggccuuugaaaguccuuacuuucaaaggccaaggcucuugcuauacccaga

SEQ ID NO: 18 - RNA, 72 nt, Artificial Sequence
  miSNCA_05 in pri-miRNA
cuugggaauggcaaggugcucuuuggucuucucagccaugagaagaccaaagagcgucuugcuauacccaga

SEQ ID NO: 19 - RNA, 72 nt, Artificial Sequence
  miSNCA_07 in pri-miRNA
cuugggaauggcaagguccaacauuugucacuugcucucaagugacaaauguuggcucuugcuauacccaga

SEQ ID NO: 20 - RNA, 72 nt, Artificial Sequence
  miSNCA_12 in pri-miRNA
cuugggaauggcaagguugguuuucucagcagcagccaugcugcugagaaaaccacucuugcuauacccaga

SEQ ID NO: 21 - RNA, 72 nt, Artificial Sequence
  miSNCA_13 in pri-miRNA
cuugggaauggcaaggaacacccucuuuugucuuuccuaagacaaaagagggugucucuugcuauacccaga

SEQ ID NO: 22 - RNA, 72 nt, Artificial Sequence
  miSNCA_15 in pri-miRNA
cuugggaauggcaagguugcccaacugguccuuuuugaaaaggaccaguugggcacucuugcuauacccaga

SEQ ID NO: 23 - RNA, 72 nt, Artificial Sequence
  miSNCA_16 in pri-miRNA
cuugggaauggcaagguuaggcuucagguucguagucuuacgaaccugaagccuacucuugcuauacccaga

SEQ ID NO: 24 - DNA, 267 bp, Artificial Sequence
  miSNCA_02 in the miR451
gatatcctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccagaaaacgtgccaggaagagaactcaggaccctgaagcagactactggaagggagactccagctcaaacaaggcaggggtgggggcgtgggatggatctcga

SEQ ID NO: 25 - DNA, 267 bp, Artificial Sequence
  miSNCA_05 in the miR451
gatatcctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggatggatctcga

SEQ ID NO: 26 - DNA, 267 bp, Artificial Sequence
  miSNCA_07 in the miR451
gatatcctctggagcctgacaaggaggacaggagagatgctgcaagcccaagaagctctctgctcagcctgtcacaacctactgactgccagggcacttgggaatggcaaggtccaacatttgtcacttgctctcaagtgacaaatgttggctcttgctatacccagaaaacgtgccaggaagagaactcaggaccctgaagcagactactggaagggagactccagctcaaacaaggcaggggtgggggcgtgggatggatctcga

SEQ ID NO: 27 - DNA, 267 bp, Artificial Sequence
  miSNCA_12 in the miR451
gatatcctctggagcctgacaaggaggacaggagagatgctgcaagcccaagaagctctctgctcagcctgtcacaacctactgactgccagggcacttgggaatggcaaggttggttttctcagcagcagccatgctgctgagaaaaccactcttgctatacccagaaaacgtgccaggaagagaactcaggaccctgaagcagactactggaagggagactccagctcaaacaaggcaggggtgggggcgtgggatggatctcga

SEQ ID NO: 28 - DNA, 267 bp, Artificial Sequence
  miSNCA_13 in the miR451
gatatcctctggagcctgacaaggaggacaggagagatgctgcaagcccaagaagctctctgctcagcctgtcacaacctactgactgccagggcacttgggaatggcaaggaacaccctcttttgtctttcctaagacaaaagagggtgtctcttgctatacccagaaaacgtgccaggaagagaactcaggaccctgaagcagactactggaagggagactccagctcaaacaaggcaggggtgggggcgtgggatggatctcga

SEQ ID NO: 29 - DNA, 267 bp, Artificial Sequence
  miSNCA_15 in the miR451
gatatcctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggatggatctcga

SEQ ID NO: 30 - DNA, 267 bp, Artificial Sequence
  miSNCA_16 in the miR451
gatatcctctggagcctgacaaggaggacaggagagatgctgcaagcccaagaagctctctgctcagcctgtcacaacctactgactgccagggcacttgggaatggcaaggttaggcttcaggttcgtagtcttacgaacctgaagcctactcttgctatacccagaaaacgtgccaggaagagaactcaggaccctgaagcagactactggaagggagactccagctcaaacaaggcaggggtgggggcgtgggatggatctcga

SEQ ID NO: 31 - DNA, 495 bp, Artificial Sequence
  miSNCA_05 in miR451 miR144
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcc

SEQ ID NO: 32 - DNA, 495 bp, Artificial Sequence
  miSNCA_15 in miR451 miR144
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcc

SEQ ID NO: 33 - DNA, 6783 bp, Artificial Sequence
  SNCA reporter plasmid
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctaggcgatcgctcgacatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtatgcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 34 - DNA, 612 bp, Artificial Sequence
  SNCA concatenated reporter
ggcgatcgctcgacatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtatgcggccgctggccgcaataaaatatctttattttcattacatctgtgtgttggttttttgtgtg

SEQ ID NO: 35 - protein, 140 aa, Artificial Sequence
  aSyn
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 36 - DNA, 423 bp, Artificial Sequence
  SNCA
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 37 - DNA, 12152 bp, Artificial Sequence
  pVD1496
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggctgcagggaggggggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcagcttgcatgcaggcctctgcagtcgacgggcccgtcgatcttgatgcaacttaattttattaggacaaggctggtgggcactggagtggcaacttccagggccaggagaggcactggggaggggtcacagggatgccacgggcggccgctcgagatccatcccacgcccccacccctgccttgtttgagctggagtctcccttccagtagtctgcttcagggtcctgagttctcttcctggcacgttttctgggtatagcaagagccttggcctttgaaagtaaggactttcaaaggccaaggaccttgccattcccaagtgccctggcagtcagtaggttgtgacaggctgagcagagagcttcttgggcttgcagcatctctcctgtcctccttgtcaggctccagaggatatctagatgcattcgcgaggtaccgagctcgaattcgcccttaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattaagggcgaattcactggccgtcgttttacaacgtcgtgactgggaaaaccctggcgttacccaacttaatcgccttgcagcacatccccctttcgccagctggcgtaatagcgaagaggcccgcaccggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacaccacaactatgcacgttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactaatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgtttttta

SEQ ID NO: 38 - DNA, 12152 bp, Artificial Sequence
  pVD1497
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggctgcagggaggggggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcagcttgcatgcaggcctctgcagtcgacgggcccgtcgatcttgatgcaacttaattttattaggacaaggctggtgggcactggagtggcaacttccagggccaggagaggcactggggaggggtcacagggatgccacgggcggccgctcgagatccatcccacgcccccacccctgccttgtttgagctggagtctcccttccagtagtctgcttcagggtcctgagttctcttcctggcacgttttctgggtatagcaagacgctctttggtcttctcatggctgagaagaccaaagagcaccttgccattcccaagtgccctggcagtcagtaggttgtgacaggctgagcagagagcttcttgggcttgcagcatctctcctgtcctccttgtcaggctccagaggatatctagatgcattcgcgaggtaccgagctcgaattcgcccttaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattaagggcgaattcactggccgtcgttttacaacgtcgtgactgggaaaaccctggcgttacccaacttaatcgccttgcagcacatccccctttcgccagctggcgtaatagcgaagaggcccgcaccggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacaccacaactatgcacgttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactaatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgtttttta

SEQ ID NO: 39 - DNA, 12152 bp, Artificial Sequence
  pVD1498
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggctgcagggaggggggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcagcttgcatgcaggcctctgcagtcgacgggcccgtcgatcttgatgcaacttaattttattaggacaaggctggtgggcactggagtggcaacttccagggccaggagaggcactggggaggggtcacagggatgccacgggcggccgctcgagatccatcccacgcccccacccctgccttgtttgagctggagtctcccttccagtagtctgcttcagggtcctgagttctcttcctggcacgttttctgggtatagcaagagccaacatttgtcacttgagagcaagtgacaaatgttggaccttgccattcccaagtgccctggcagtcagtaggttgtgacaggctgagcagagagcttcttgggcttgcagcatctctcctgtcctccttgtcaggctccagaggatatctagatgcattcgcgaggtaccgagctcgaattcgcccttaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattaagggcgaattcactggccgtcgttttacaacgtcgtgactgggaaaaccctggcgttacccaacttaatcgccttgcagcacatccccctttcgccagctggcgtaatagcgaagaggcccgcaccggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacaccacaactatgcacgttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactaatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgtttttta

SEQ ID NO: 40 - DNA, 12152 bp, Artificial Sequence
  pVD1499
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggctgcagggaggggggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcagcttgcatgcaggcctctgcagtcgacgggcccgtcgatcttgatgcaacttaattttattaggacaaggctggtgggcactggagtggcaacttccagggccaggagaggcactggggaggggtcacagggatgccacgggcggccgctcgagatccatcccacgcccccacccctgccttgtttgagctggagtctcccttccagtagtctgcttcagggtcctgagttctcttcctggcacgttttctgggtatagcaagagtggttttctcagcagcatggctgctgctgagaaaaccaaccttgccattcccaagtgccctggcagtcagtaggttgtgacaggctgagcagagagcttcttgggcttgcagcatctctcctgtcctccttgtcaggctccagaggatatctagatgcattcgcgaggtaccgagctcgaattcgcccttaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattaagggcgaattcactggccgtcgttttacaacgtcgtgactgggaaaaccctggcgttacccaacttaatcgccttgcagcacatccccctttcgccagctggcgtaatagcgaagaggcccgcaccggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacaccacaactatgcacgttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactaatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgtttttta

SEQ ID NO: 41 - DNA, 12152 bp, Artificial Sequence
  pVD1500
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggctgcagggaggggggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcagcttgcatgcaggcctctgcagtcgacgggcccgtcgatcttgatgcaacttaattttattaggacaaggctggtgggcactggagtggcaacttccagggccaggagaggcactggggaggggtcacagggatgccacgggcggccgctcgagatccatcccacgcccccacccctgccttgtttgagctggagtctcccttccagtagtctgcttcagggtcctgagttctcttcctggcacgttttctgggtatagcaagagacaccctcttttgtcttaggaaagacaaaagagggtgttccttgccattcccaagtgccctggcagtcagtaggttgtgacaggctgagcagagagcttcttgggcttgcagcatctctcctgtcctccttgtcaggctccagaggatatctagatgcattcgcgaggtaccgagctcgaattcgcccttaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattaagggcgaattcactggccgtcgttttacaacgtcgtgactgggaaaaccctggcgttacccaacttaatcgccttgcagcacatccccctttcgccagctggcgtaatagcgaagaggcccgcaccggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacaccacaactatgcacgttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactaatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgtttttta

SEQ ID NO: 42 - DNA, 12152 bp, Artificial Sequence
  pVD1501
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggctgcagggaggggggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcagcttgcatgcaggcctctgcagtcgacgggcccgtcgatcttgatgcaacttaattttattaggacaaggctggtgggcactggagtggcaacttccagggccaggagaggcactggggaggggtcacagggatgccacgggcggccgctcgagatccatcccacgcccccacccctgccttgtttgagctggagtctcccttccagtagtctgcttcagggtcctgagttctcttcctggcacgttttctgggtatagcaagagtgcccaactggtccttttcaaaaaggaccagttgggcaaccttgccattcccaagtgccctggcagtcagtaggttgtgacaggctgagcagagagcttcttgggcttgcagcatctctcctgtcctccttgtcaggctccagaggatatctagatgcattcgcgaggtaccgagctcgaattcgcccttaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattaagggcgaattcactggccgtcgttttacaacgtcgtgactgggaaaaccctggcgttacccaacttaatcgccttgcagcacatccccctttcgccagctggcgtaatagcgaagaggcccgcaccggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacaccacaactatgcacgttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactaatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgtttttta

SEQ ID NO: 43 - DNA, 12152 bp, Artificial Sequence
  pVD1502
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggctgcagggaggggggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcagcttgcatgcaggcctctgcagtcgacgggcccgtcgatcttgatgcaacttaattttattaggacaaggctggtgggcactggagtggcaacttccagggccaggagaggcactggggaggggtcacagggatgccacgggcggccgctcgagatccatcccacgcccccacccctgccttgtttgagctggagtctcccttccagtagtctgcttcagggtcctgagttctcttcctggcacgttttctgggtatagcaagagtaggcttcaggttcgtaagactacgaacctgaagcctaaccttgccattcccaagtgccctggcagtcagtaggttgtgacaggctgagcagagagcttcttgggcttgcagcatctctcctgtcctccttgtcaggctccagaggatatctagatgcattcgcgaggtaccgagctcgaattcgcccttaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcagggggggttgcagcccgaccgacgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcggagccgcacgcggcgctccccacgcacacacacacgcacgcaccccccgagccgctccccccgcacaaagggccctcccggagcccctcaaggctttcacgcagccacagaaaagaaacaagccgtcattaaaccaagcgctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggtaccgaattaagggcgaattcactggccgtcgttttacaacgtcgtgactgggaaaaccctggcgttacccaacttaatcgccttgcagcacatccccctttcgccagctggcgtaatagcgaagaggcccgcaccggatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacaccacaactatgcacgttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactaatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgtttttta

SEQ ID NO: 44 - DNA, 133 bp, Artificial Sequence
  SV40 polyA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 45 - DNA, 269 bp, Artificial Sequence
  BGH polyA
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 46 - RNA, 16 nt, Artificial Sequence
  First flanking miR451
cuugggaauggcaagg

SEQ ID NO: 47 - RNA, 17 nt, Artificial Sequence
  Second flanking  miR451 (M is a G or a C and W is an A or a U)
mwcuugcuauacccaga

SEQ ID NO: 48 - DNA, 20 bp, Artificial Sequence
  SNCA1-Forward primer
aaatgttggaggagcagtgg

SEQ ID NO: 49 - DNA, 20 bp, Artificial Sequence
  SNCA1-Reverse primer
tccagaattccttcctgtgg

SEQ ID NO: 50 - DNA, 21 bp, Artificial Sequence
  UBE-Forward primer
tgcctgagattgctcggatct

SEQ ID NO: 51 - DNA, 22 bp, Artificial Sequence
  UBE Reverse primer
tcgcatacttctgagtccattc

SEQ ID NO: 52 - DNA, 23 bp, Artificial Sequence
  CYC1 Forward primer
agcctacaagaaagtttgcctat

SEQ ID NO: 53 - DNA, 22 bp, Artificial Sequence
  CYC1 Reverse primer
tcttcttccggtagtggatctt

SEQ ID NO: 54 - DNA, 18 bp, Artificial Sequence
  ACTB Forward primer
gtcttcccctccatcgtg

SEQ ID NO: 55 - DNA, 18 bp, Artificial Sequence
  ACTB Reverse primer
tcttgctctgggcctcgt

SEQ ID NO: 56 - DNA, 20 bp, Artificial Sequence
  SNCA2 Forward primer
atgttggaggagcagtggtg

SEQ ID NO: 57 - DNA, 20 bp, Artificial Sequence
  SNCA Reverse primer
tgtcaggatccacaggcata

SEQ ID NO: 58 - DNA, 20 bp, Artificial Sequence
  GFP Forward primer
agcaaagaccccaacgagaa

SEQ ID NO: 59 - DNA, 17 bp, Artificial Sequence
  GFP Reverse primer
gcggcggtcacgaactc

SEQ ID NO: 60 - DNA, 18 bp, Artificial Sequence
  B2M Forward primer
gccatccaccggagaatg

SEQ ID NO: 61 - DNA, 22 bp, Artificial Sequence
  B2M Reverse primer
ggtggaactgagacacgtagca

SEQ ID NO: 62 - DNA, 19 bp, Artificial Sequence
  GAPDH Forward primer
tgcccccatgtttgtgatg

SEQ ID NO: 63 - DNA, 23 bp, Artificial Sequence
  GAPDH Reverse primer
gctgacaatcttgagggagttgt

SEQ ID NO: 64 - DNA, 20 bp, Artificial Sequence
  ACTB Forward primer
agcgtggctacagcttcacc

SEQ ID NO: 65 - DNA, 24 bp, Artificial Sequence
  ACTB Reverse primer
aagtctagggcaacatagcacagc

SEQ ID NO: 66 - DNA, 21 bp, Artificial Sequence
  HPRT Forward primer
gcgaaagtggaaaagccaagt

SEQ ID NO: 67 - DNA, 23 bp, Artificial Sequence
  HPRT Reverse primer
gccacatcaacaggactcttgta

SEQ ID NO: 68 - DNA, 21 bp, Artificial Sequence
  SNCA1 Probe
agcagggagcattgcagcagc

SEQ ID NO: 69 - DNA, 23 bp, Artificial Sequence
  SNCA2 Probe
cagcagccactggctttgtcaaa

SEQ ID NO: 70 - DNA, 20 bp, Artificial Sequence
  SNCA1 Forward primer
gactttcaaaggccaaggag

SEQ ID NO: 71 - DNA, 20 bp, Artificial Sequence
  SNCA1 Reverse primer
ggagcctacatagagaacac

SEQ ID NO: 72 - DNA, 22 bp, Artificial Sequence
  SNCA2 Forward primer
attgcagcagccactggctttg

SEQ ID NO: 73 - DNA, 21 bp, Artificial Sequence
  SNCA2 Reverse primer
ggcttcaggttcgtagtcttg

SEQ ID NO: 74 - DNA, 24 bp, Artificial Sequence
  PMP3 Forward primer
cactcatctctatgacgacgtttc

SEQ ID NO: 75 - DNA, 20 bp, Artificial Sequence
  PMP3 Reverse primer
caccgtcgagaagctgtaga

SEQ ID NO: 76 - DNA, 381 bp, Artificial Sequence
  SNCA126
atggatgtattcatgaaaggactttcaaaggccaaggagggagttgtggctgctgctgagaaaaccaaacagggtgtggcagaagcagcaggaaagacaaaagagggtgttctctatgtagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 77 - DNA, 339 bp, Artificial Sequence
  SNCA112
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaa

SEQ ID NO: 78 - DNA, 297 bp, Artificial Sequence
  SNCA98
atggatgtattcatgaaaggactttcaaaggccaaggagggagttgtggctgctgctgagaaaaccaaacagggtgtggcagaagcagcaggaaagacaaaagagggtgttctctatgtagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaa

SEQ ID NO: 79 - DNA, 101 bp, Artificial Sequence
  hGH polyA
cccgtggcatccctgtgacccctccccagtgcctctcctggccctggaagttgccactccagtgcccaccagccttgtcctaataaaattaagttgcatca

SEQ ID NO: 80 - DNA, 264 bp, Artificial Sequence
  miSNCA1
gatatcctctggagcctgacaaggaggacaggagagatgctgcaagcccaagaagctctctgctcagcctgtcacaacctactgactgccagggcacttgggaatggcaaggcctttgaaagtcctttcatgaatgaaaggactttcaaagctcttgctatacccagaaaacgtgccaggaagagaactcaggaccctgaagcagactactggaagggagactccagctcaaacaaggcaggggtgggggcgtgggatggatcc

SEQ ID NO: 81 - DNA, 264 bp, Artificial Sequence
  miSNCA3
gatatcctctggagcctgacaaggaggacaggagagatgctgcaagcccaagaagctctctgctcagcctgtcacaacctactgactgccagggcacttgggaatggcaaggttctcagcagcagccacaactctgtggctgctgctgagactcttgctatacccagaaaacgtgccaggaagagaactcaggaccctgaagcagactactggaagggagactccagctcaaacaaggcaggggtgggggcgtgggatggatcc

SEQ ID NO: 82 - DNA, 264 bp, Artificial Sequence
  miSNCA4
gatatcctctggagcctgacaaggaggacaggagagatgctgcaagcccaagaagctctctgctcagcctgtcacaacctactgactgccagggcacttgggaatggcaaggctacatagagaacaccctctttagggtgttctctatgtactcttgctatacccagaaaacgtgccaggaagagaactcaggaccctgaagcagactactggaagggagactccagctcaaacaaggcaggggtgggggcgtgggatggatcc

SEQ ID NO: 83 - DNA, 264 bp, Artificial Sequence
  miSNCA6
gatatcctctggagcctgacaaggaggacaggagagatgctgcaagcccaagaagctctctgctcagcctgtcacaacctactgactgccagggcacttgggaatggcaagggtcacttgctctttggtcttctgaccaaagagcaagtgaatcttgctatacccagaaaacgtgccaggaagagaactcaggaccctgaagcagactactggaagggagactccagctcaaacaaggcaggggtgggggcgtgggatggatcc

SEQ ID NO: 84 - DNA, 264 bp, Artificial Sequence
  miSNCA9
gatatcctctggagcctgacaaggaggacaggagagatgctgcaagcccaagaagctctctgctcagcctgtcacaacctactgactgccagggcacttgggaatggcaaggcttctgggctactgctgtcacaacagcagtagcccagaactcttgctatacccagaaaacgtgccaggaagagaactcaggaccctgaagcagactactggaagggagactccagctcaaacaaggcaggggtgggggcgtgggatggatcc

SEQ ID NO: 85 - DNA, 264 bp, Artificial Sequence
  miSNCA10
gatatcctctggagcctgacaaggaggacaggagagatgctgcaagcccaagaagctctctgctcagcctgtcacaacctactgactgccagggcacttgggaatggcaaggactgtcttctgggctactgctgagtagcccagaagacagctcttgctatacccagaaaacgtgccaggaagagaactcaggaccctgaagcagactactggaagggagactccagctcaaacaaggcaggggtgggggcgtgggatggatcc

SEQ ID NO: 86 - DNA, 264 bp, Artificial Sequence
  miSNCA11
gatatcctctggagcctgacaaggaggacaggagagatgctgcaagcccaagaagctctctgctcagcctgtcacaacctactgactgccagggcacttgggaatggcaaggcacattggaactgagcacttgtgtgctcagttccaatgtcacttgctatacccagaaaacgtgccaggaagagaactcaggaccctgaagcagactactggaagggagactccagctcaaacaaggcaggggtgggggcgtgggatggatcc

SEQ ID NO: 87 - DNA, 264 bp, Artificial Sequence
  miSNCA14
gatatcctctggagcctgacaaggaggacaggagagatgctgcaagcccaagaagctctctgctcagcctgtcacaacctactgactgccagggcacttgggaatggcaaggggttcgtagtcttgatacccttgtatcaagactacgaacatcttgctatacccagaaaacgtgccaggaagagaactcaggaccctgaagcagactactggaagggagactccagctcaaacaaggcaggggtgggggcgtgggatggatcc

SEQ ID NO: 88 - DNA, 264 bp, Artificial Sequence
  miSNCA18
gatatcctctggagcctgacaaggaggacaggagagatgctgcaagcccaagaagctctctgctcagcctgtcacaacctactgactgccagggcacttgggaatggcaaggctccttggttttggagcctacaggctccaaaaccaaggactcttgctatacccagaaaacgtgccaggaagagaactcaggaccctgaagcagactactggaagggagactccagctcaaacaaggcaggggtgggggcgtgggatggatcc

SEQ ID NO: 89 - DNA, 264 bp, Artificial Sequence
  miSNCA19
gatatcctctggagcctgacaaggaggacaggagagatgctgcaagcccaagaagctctctgctcagcctgtcacaacctactgactgccagggcacttgggaatggcaaggctttgagaaatgtcatgactggtcatgacatttctcaaactcttgctatacccagaaaacgtgccaggaagagaactcaggaccctgaagcagactactggaagggagactccagctcaaacaaggcaggggtgggggcgtgggatggatcc

SEQ ID NO: 90 - DNA, 267 bp, Artificial Sequence
  miSCR
gatatcctctggagcctgacaaggaggacaggagagatgctgcaagcccaagaagctctctgctcagcctgtcacaacctactgactgccagggcacttgggaatggcaaggtctacatcgtctctatctcttcagatagagacgatgtagctcttgctatacccagaaaacgtgccaggaagagaactcaggaccctgaagcagactactggaagggagactccagctcaaacaaggcaggggtgggggcgtgggatggatctcga

SEQ ID NO: 91 - DNA, 4811 bp, Artificial Sequence
  pVD1587
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgagcggaaggcccatgaggccagttaattaacgagctcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaagtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgcgccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggggcggccgcaagctttagagctcatggcgcgcctaggccttgacggccttccgccaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 92 - DNA, 4811 bp, Artificial Sequence
  pVD1588
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgagcggaaggcccatgaggccagttaattaacgagctcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaagtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgcgccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggggcggccgcaagctttagagctcatggcgcgcctaggccttgacggccttccgccaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

(19) United States
(12) Patent Application Publication
(10) Pub. No.: US 2025/0382611 A1
(43) Pub. Date: Dec. 18, 2025

(54) Gene constructs for silencing alpha-synuclein and uses thereof

(71) Applicant: uniQure biopharma B.V., Amsterdam (NL)

(72) Inventors: Seyda Acar Broekmans, Amsterdam (NL); Astrid Valles Sanchez, Amsterdam (NL)

(73) Assignee: uniQure biopharma B.V., Amsterdam (NL)

(21) Appl. No.: 18/568,860

(22) PCT Filed: Jun. 21, 2022
(86) PCT No.: PCT/EP2022/066922

§ 371 Date: Dec. 11, 2023

(30) Foreign Application Priority Data
Jun. 21, 2021 (EP) 21180613.8
Apr. 12, 2022 (EP) 22168018.4

(51) Int. Cl. C12N 15/113; A61K 31/713; A61P 25/16; C12N 15/86

(52) U.S. Cl. CPC C12N 15/113; A61K 31/713; A61P 25/16; C12N 15/86; C12N 2310/141; C12N 2310/531; C12N 2750/14122; C12N 2750/14143

(57) ABSTRACT

The present invention relates to a nucleic acid, to a composition, to the medical use of said composition in the treatment and/or prevention of Parkinson's Disease (PD), Multiple System Atrophy (MSA) and/or other α-synucleopathies.

### TECHNICAL FIELD

The present invention relates to a nucleic acid, to the use of said nucleic acid for decreasing and/or knocking down the transcripts of alpha-synuclein (α-syn) gene (SNCA) and to treat and/or prevent Parkinson's disease (PD) and other α-synucleopathies, particularly in a gene therapy setting.

### BACKGROUND OF THE INVENTION

Fibrillar α-synuclein inclusion bodies define two major classes of neurodegenerative disease: Lewy body diseases, including PD, Lewy body dementia (LBD), which includes PD with dementia (PDD), and dementia with Lewy bodies (DLB)), and those characterized by Papp-Lantos bodies, including multiple system atrophy (MSA). These are collectively termed synucleinopathies. Parkinson's disease (PD) is a complex progressive neurodegenerative disorder, which can cause motor and non-motor symptoms. The typical clinical features of PD comprise bradykinesia, resting tremor, rigidity, and/or postural instability occurring at a later stage. Before and/or after clinical diagnosis, non-motor symptoms can take place. These include depression, sleep disturbances, pain and fatigue at earlier stages of the disease, and anxiety, dementia and cognitive dysfunction at later disease stages. Both motor and non-motor symptoms are debilitating for patients and create a burden to their caretakers.

PD is a complex disease, the causes of which remain unclear, although a number of genes have been found to be involved in the cause and/or development of PD. The main hallmark of PD pathology is the neurodegeneration of dopaminergic neurons in the substantia nigra, which is a mesencephalic brain region with relevant dopaminergic projections to the striatum and cortex, central for motor-related functions. In addition to the loss of nigrostriatal dopaminergic innervation and degeneration in other brain regions, PD is characterized by the presence of cytoplasmic protein aggregates (Lewy bodies) which contain insoluble α-syn proteins.

Native α-syn protein in the brain is mostly unfolded, without a defined tertiary structure. Upon interaction with negatively charged lipids, such as the phospholipids that make up cell membranes, α-syn folds into α-helical structures through its N-terminal end. In PD however, α-syn adopts a β-sheet-rich amyloid-like structure that is prone to aggregate. The aggregates constitute a major part in Lewy bodies.

MSA is a progressive, adult-onset neurodegenerative disorder of undetermined aetiology characterized by a distinctive oligodendrogliopathy with argyrophilic glial cytoplasmic inclusions (GCIs) and selective neurodegeneration. GCIs or Papp-Lantos inclusions/bodies are now accepted as the hallmarks for the definite neuropathological diagnosis of MSA and suggested to play a central role in the pathogenesis of this disorder. GCIs are composed of hyperphosphorylated α-syn, ubiquitin, LRRK2 (leucin-rich repeat serine/threonine-protein) and other proteins.

Generally speaking, α-syn proteins are prone to form aggregates, and these aggregates can result in loss of normal function and/or toxic effects in neurons, which consequently cause neurodegeneration and/or neuroinflammation in different brain areas. Further, it is known that mutations or duplications/triplications of the α-syn gene are linked to α-synucleopathies.

Presently, therapies for treating and/or preventing a disease are based on completely knocking down a gene and/or transcripts of a gene. However, because of the important physiological role of α-syn, the depletion of α-syn proteins may bring patient's safety concerns due to phenomena such as attenuated synaptic transmission in the central nervous system (CNS).

Hence, there remains a need for having a therapy which can treat and/or prevent PD and/or other synucleinopathies while reducing and/or preventing unwanted safety risks.

### SUMMARY OF THE INVENTION

A first aspect of the invention relates to a nucleic acid (“nucleic acid of the invention”) comprising a nucleic acid sequence encoding an RNA (“RNA of the invention”), wherein an RNA sequence comprised in said RNA is substantially complementary to a target sequence of an alpha-synuclein (α-syn) gene (SNCA), wherein said RNA sequence has at least 15 nucleotides, wherein said RNA includes a hairpin.

A second aspect of the invention relates to the nucleic acid of the invention which is a DNA molecule (“DNA molecule of the invention”).

A third aspect of the invention relates to an adeno-associated virus (AAV) vehicle comprising the DNA molecule (“AAV (vehicle) of the invention”).

Further aspects of the invention relate to a composition comprising the AAV vehicle of the invention and at least one pharmaceutically acceptable excipient; a method for producing the AAV vehicle of the invention; and a kit comprising the AAV vehicle if the invention, wherein said kit further comprises an immunosuppressive compound.

### DETAILED DESCRIPTION OF THE INVENTION

The present invention relates to gene therapy, in particular, to the use of RNA interference (RNAi) in gene therapy for targeting RNA encoded by the α-syn gene, preferably by the human α-syn gene.

### Nucleic Acids

According to the present invention, a nucleic acid is provided (“nucleic acid of the invention”) that comprises a nucleic acid sequence encoding an RNA (“RNA of the invention”), wherein an RNA sequence comprised in said RNA is substantially complementary to a target sequence of an α-syn gene, wherein said RNA sequence has at least 15 nucleotides, and wherein said RNA includes a hairpin.

The term “substantially complementary”, as used herein, refers to two nucleic acid sequences being complementary to each other, and thereby the two nucleic acid sequences bind to each other. The term “substantially” means that the complementarity between the two sequences is sufficient to bind to each other for an amount of time sufficient to have an at least partial inhibitory effect. It is preferred of course that the complementarity is complete (full complementarity), but some gaps and/or mismatches may be allowed. The number of mismatches should be no higher than 10%. The important feature is that the complementarity is sufficient to allow for binding of the two strands in situ. The binding must be strong enough to exert an inhibitory effect.

Said nucleic acid sequence encoding the RNA as described above optionally has at most: 4 nucleotides; 5 nucleotides; or 6 nucleotides different from a complementary (“anti”) sequence of said target sequence. Optionally, said nucleic acid sequence encoding the RNA has 1 nucleotide, 2 nucleotides, or 3 nucleotides different from a complementary sequence of said target sequence encoded by the α-syn gene. Preferably, said nucleic acid sequence as described above is identical to a complementary sequence of said target sequence.

The term “α-syn gene” as used herein, refers to an alpha-symiclein gene or SNCA gene. Said α-syn gene, as described herein, is preferably a mammalian α-syn gene, still preferably a mouse or a rat α-syn gene, more preferably a NHP α-syn gene, and most preferably a human α-syn gene. All SNPs of α-syn gene can be further included in the present invention.

The term “α-syn protein” as used herein, refers to proteins encoded by α-syn gene.

Typically the nucleic acids according to the invention are intended to diminish the expression of a disease related gene. According to the invention, said nucleic acid, as described above, can be delivered into a target cell, for example by a gene delivery vehicle, in particular a viral gene delivery vehicle preferably an adeno-associated virus (AAV) vehicle, as described below. Said nucleic acid may subsequently be transcribed into an RNA. In the process of RNA intervention (RNAi), said RNA is cleaved by Drosha (i.e. a class 2 ribonuclease III enzyme) into a short hairpin RNA (shRNA) or a long hairpin RNA (lhRNA) in the nucleus of the target cell without the flanking regions at the 5′ and 3′ ends of the RNA. Subsequently, the cleaved RNA is exported to the cytoplasm of the cell, wherein said cleaved RNA is not further cleaved by an endoribonuclease Dicer. Said cleaved RNA is further cleaved by Argonaute-2 (AGO-2) of the RNA-induced silencing complex (RISC), wherein the passenger RNA sequence of said cleaved RNA is trimmed off (i.e. cleaved) by poly (A)-specific ribonuclease (PARN). The other strand of said cleaved RNA is called a guide strand (i.e. a guide sequence). The guide strand comprising the sequence substantially complementary to said target RNA sequence, as described above, is not processed and/or cleaved by AGO-2.

In the situation that a passenger strand of a cleaved RNA remains present without being trimmed off, said passenger strand can be partially complementary to an off-target sequence and/or even to a target sequence. Hence, said passenger strand can bind to the off-target sequence and/or even compete with the guide strand of the cleaved RNA to bind to said target sequence. Such “off-target issue” can affect the precision of gene-editing intervention, and thereby has to be reduced and/or eliminated.

Thereby, cleaving said passenger sequence can prevent and/or inhibit the “off-target issue”. Hence, the binding specificity of said guide sequence to the target mRNA is improved, and the “off-target” events are reduced. This is a preferred embodiment of the invention.

An RNA comprising two strands that are complementary to each other and of which one of the strands (passenger strand) is cleaved in the RNAi is included in the present invention. For example, double-stranded RNA (dsRNA), small interfering RNA (siRNA), and microRNA (miRNA) are included in the present invention.

The term “RNA hairpin” or “hairpin”, as described herein, refers to a secondary structure of an RNA, which comprises two strands complementary to each other and a loop which connects the two strands. One of the strands is called passenger strand (i.e. passenger sequence), and the other one is called guide strand (i.e. guide sequence). An RNA hairpin can guide RNA folding, determine interactions in a ribozyme, protect messenger RNA (mRNA) from degradation, and serve as a recognition motif for RNA binding protein.

Other RNAs with two strands are also included in the present invention, provided that preferably one of the strands is degraded (i.e. trimmed off) in RNA interference (RNAi) while the other strand remains without being degraded, and that said “off-target issue” is improved. A lhRNA and/or a shRNA can be included in the present invention. In certain embodiments, said hairpin may be shRNA or lhRNA.

Preferably, said hairpin as described above, has a sequence of at least 39 nucleotides; at least 44 nucleotides; at least 49 nucleotides; at least 54 nucleotides; or at least 59 nucleotides. In some embodiments of the invention, the hairpin as described above has a sequence of at least 39 nucleotides. Thus, preferably, the nucleic acid sequence encoding the RNA has a sequence of at least 39 nucleotides.

Optionally, said hairpin as described above, has a RNA sequence of at most 80 nucleotides, optionally at most 78 nucleotides, optionally at most 76 nucleotides, optionally at most 74 nucleotides, optionally at most 72 nucleotides, optionally at most 70 nucleotides, optionally at most 68 nucleotides, optionally at most 66 nucleotides, and still optionally at most 64 nucleotides. Preferably, said hairpin as described above, has a RNA sequence of 72 nucleotides.

miRNA Scaffolds

A nucleic acid sequence encoding said hairpin having said sequence length as described above, can be easily incorporated in an AAV, and be delivered to a target organ, such as the central nervous system. Further, said lengths allow said hairpin to be folded correctly, so that said passenger strand can be cleaved in the RNAi as described above. Therefore, said sequences having said lengths, as described above, can reduce and/or prevent said off-target issues. Furthermore, said off-target issues are further reduced and/or prevented through an RNA which has a sequence selected from the group consisting of: SEQ ID NO.1, SEQ ID NO.2, and variants of SEQ ID NO.1 and SEQ ID NO. 2.

Thus, in preferred embodiments, said RNA (the RNA of the invention) comprises SEQ ID NO.1, SEQ ID NO.2, or a variant of SEQ ID NO.1, or SEQ ID NO. 2.

Therefore, in preferred embodiments, the invention provides a nucleic acid comprising a nucleic acid sequence encoding an RNA, wherein an RNA sequence comprised in said RNA is substantially complementary to a target sequence of an α-syn gene, wherein said RNA sequence has at least 15 nucleotides, wherein said RNA includes a hairpin and wherein said RNA comprises SEQ ID NO.1, SEQ ID NO.2, or a variant of SEQ ID NO.1 or SEQ ID NO.2.

SEQ ID NO.1 refers to a miR451 scaffold or hairpin. Said scaffold preferably comprises from 5′ to 3′, firstly (i) 5′-CUUGGGAAUGGCAAGG-3′ (SEQ ID NO.46), followed by (ii) a sequence of 22 nucleotides, comprising or consisting of a first RNA sequence, followed by (iii) a sequence of 17 nucleotides, which can be regarded as a second RNA sequence, which is complementary over its entire length with nucleotides 2-18 of said first sequence of 22 nucleotides, subsequently followed by (iv) sequence 5′-MWCUUGCUAUACCCAGA-3′ (wherein M is a G or a C and W is an A or a U) (SEQ ID NO.47). Preferably the first 5′-A/C nucleotide of the latter sequence is not to base pair with the first nucleotide of the first strand of the first or second RNA.

Such a scaffold may comprise flanking sequences as found in the original pri-miR451 scaffold. Alternatively, the flanking sequences may be replaced by flanking sequences of other pri-mRNA structures. pri-mRNA sequences of exemplary scaffolds of the invention are provided in Table 3.

The miR451 scaffold allows to induce RNA interference (RNAi); particularly, the RNAi is induced by the guide strand of this scaffold. The pri-miR451 scaffold does not result in a passenger strand because the processing is different from the canonical miRNA processing pathway (Cheloufi, S. et. al., 2010 and Yang, J. S. et. al., 2010). Thereby, the use of miR-451 can prevent or reduce the possibility of having unwanted potential off-targeting by passenger strands.

SEQ ID NO.2 refers to a miR-144 scaffold combined with a miR451 scaffold as described above.

Said nucleic acid can be transcribed into said RNA, as described above. Preferably, the RNA as described above comprises a hairpin of miR-451 which comprises SEQ ID NO.1. With the use of said miR451, the off-target issues as described above are prevented and/or reduced, because said passenger strand is cleaved and not present in the final miR451. More preferably, said RNA comprises SEQ ID NO.2 and has a double hairpin structure. Said structure comprises a hairpin miR144 followed by said hairpin miR451 from 5′ end to 3′ end of said RNA. It was found that when said RNA comprises SEQ ID NO. 2, said off-target issues are prevented and/or reduced. Furthermore, the biogenesis of said hairpin miR451 is improved, and thereby the amount of said guide strands increases. Hence, the inhibition and/or knock off the transcripts of the target RNA can be enhanced.

An RNA variant of SEQ ID NO. 1 or SEQ ID NO. 2 is defined as having substantially the same functions as the RNA comprising SEQ ID NO. 1 or SEQ ID NO. 2, respectively. The RNA comprising said variant of SEQ ID NO. 1 or SEQ ID NO. 2 has the function of preventing and/or reducing said off-target issues as described above. Said variants of SEQ ID NO.1 and SEQ ID NO.2 also have substantially the same function as SEQ ID. NO. 1 and SEQ ID. NO. 2, respectively, for folding to a RNA secondary structure. Moreover, the RNA comprising said variant of said SEQ ID NO. 2 can not only reduce and/or prevent said off-target issues, but also improve the biogenesis of said hairpin, as described above.

When describing said “off-target issue” as reduced/improved, as described herein, it is meant that said off-target issue is prevented, reduced and/or stopped.

Optionally, said variant of SEQ ID NO.1 as described above is substantially the same as SEQ ID NO. 1, and has substantially the same function as SEQ ID NO. 1 as described above. Optionally, said variant comprises at least one nucleotide, or optionally at most 5 nucleotides different from SEQ ID NO. 1. Optionally, said variant of SEQ ID NO. 1 comprises at most 30 nucleotides; at most 25 nucleotides; at most 20 nucleotides; at most 15 nucleotides; or at most 10 nucleotides different from SEQ ID NO. 1.

Optionally, a variant of SEQ ID NO. 2 as described above is substantially the same as SEQ ID NO. 2 and has substantially the same function as SEQ ID NO. 2 as described above. Optionally, said variant can comprise at least one nucleotide or, optionally, at most 5 nucleotides different from SEQ ID NO. 2. Optionally, said variant of SEQ ID NO.2 comprises at most 30 nucleotides; at most 25 nucleotides; at most 20 nucleotides; at most 15 nucleotides; or at most 10 nucleotides different from SEQ ID NO.2.

Preferably, said RNA sequence substantially complementary to said target RNA sequence encoded by the α-syn gene has at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, or at least 24 nucleotides. Preferably, said RNA sequence as described herein has at least 18 nucleotides.

Optionally, said RNA sequence has at most 32 nucleotides, at most 31 nucleotides, at most 30 nucleotides, at most 29 nucleotides, at most 28 nucleotides, at most 27 nucleotides, at most 26 nucleotides, or at most 25 nucleotides. In some embodiments of the invention, said RNA sequence has at most 32 nucleotides. Thus, the nucleic acid sequence encoding said RNA has at most 32 nucleotides.

Said RNA sequence having the sequence length as described above, constitutes the guide strand of said hairpin as described above. As described above, the length of said guide strand is designed to form the guide strand of said hairpin and to allow the RNA secondary structure (i.e. hairpin) to form. Also, the length of said guide strand is selected for providing a sufficient binding specificity to said target RNA. These contribute to reducing said off-target issues.

The RNA sequence substantially complementary to a target sequence of the α-syn gene (said sequence comprised in said DNA) is designed based on one of the conserved regions in the α-syn gene, as described below.

Preferably, said conserved regions are present in the mammalian α-syn gene, more preferably in the non-human primate (NHP), and/or human α-syn gene.

Preferably, said target RNA is encoded by a part of an exon comprised in said α-syn gene. Since exons are not removed by RNA splicing, exons are useful to take into account when designing said target RNA.

The term “(a) part of” as defined herein, refers to a partial sequence. The term “exon” as defined herein, refers to a region comprised in said α-syn gene which encodes a part of a mRNA without being removed by RNA splicing. An exon can comprise at least one conserved sequence. Exons comprised in the NHP and human α-syn genes were aligned for designing said target RNAs and said guide strands. For instance, said NHP α-syn gene consists of the NHP α-syn gene (gene ID: 706985, https://www.ncbi.nlm.nih.gov/gene/706985). For example, said human α-syn gene consists of the human α-syn gene (Gene ID: 6622 (https://www.ncbi.nlm.nih.gov/gene/6622)).

The term “at least one” as described herein, refers to that an indicated subject, such as a conserved sequence, as described herein, is in the amount of one, two, three, or more.

The term “conserved sequence” or “conserved region”, as described herein, refers to a short length of sequence which can be found in various species with a high level of similarity. A conserved sequence can be identified through aligning a number of nucleic acid sequences from various species for encoding an RNA or a protein having similar functions, and thereby a part of or majority of the sequences are identical.

Each of exon 2, exon 3, exon 4, exon 5, and exon 6 in the α-syn gene comprises at least one conserved region for designing a target RNA to which said guide strand as described above can bind. Preferably, said exon is selected from the group consisting of exon 2, exon 4, and exon 6. It has been found that multiple conserved sequences are present in exons 2, 4, and 6 of the NHP α-syn gene and/or human α-syn gene. Hence, said exons are useful in designing said RNA.

Preferably, said guide strand binds to said target RNA encoded by part of exon 2 or exon 4, and more preferably, by a part of exon 4. In other words, the target RNA sequence is part of exon 2, exon 4 or exon 6; preferably part of exon 2 or 4; and more preferably part of exon 4.

The transcripts of said target RNA designed based on the conserved sequences in exon 2, exon 4, and/or exon 6 can be reduced and/or knocked down by said guide strands as described below.

The term “transcripts” as described here, refers to mRNA, proteins and/or protein aggregates encoded by the α-syn gene. The term “α-syn aggregates”, “aggregates of the α-syn proteins”, and/or other variants, as described herein, refers to aggregates composed of α-syn proteins.

Any exon, such as exon 3 and/or exon 5, that is included in the α-syn gene and that comprises at least a conserved sequence is also included in the present invention.

More preferably, said part of said exon, as described above, is selected from a group consisting of SEQ ID NOs 3-9 (Table 1) and variants of SEQ ID NOs 3-9, preferably consisting of SEQ ID NO. 4, 7 and 8 and variants of SEQ ID NO. 4, 7 and 8, more preferably consisting of SEQ ID NO. 4 and 8 and variants of SEQ ID NO. 4 and 8. In other words, said part of said exon consists of a sequence selected from the group consisting of SEQ ID NO. 3 to 9 and variants of SEQ ID NO. 3 to 9.

TABLE 1
Suitable target RNA sequence of the present invention

| | Sequence | | |
|---|---|---|---|
| SEQ ID | targeted by | Target RNA Sequence | Length |
| NO. | miSNCA | (5′-sequence-3′) | (nucleotides) |
| 3 | 02 | AAGGACTTTCAAAGGCCAAGGA | 22 |
| 4 | 05 | TGGCTGAGAAGACCAAAGAGCA | 22 |
| 5 | 07 | AGAGCAAGTGACAAATGTTGGA | 22 |
| 6 | 12 | TGGCTGCTGCTGAGAAAACCAA | 22 |
| 7 | 13 | AGGAAAGACAAAAGAGGGTGTT | 22 |
| 8 | 15 | TCAAAAAGGACCAGTTGGGCAA | 22 |
| 9 | 16 | AGACTACGAACCTGAAGCCTAA | 22 |

Said variants of SEQ ID NOs 3-9 have substantially the same sequences and functions as SEQ ID NOs 3-9, respectively. Said variants can be bound by a guide strand, as described below, and subsequently said target RNA and its transcripts, such as proteins, are reduced and/or knocked down. Said variants of SEQ ID NOs 3-9 have at least one nucleotide and at most 5 nucleotides different from SEQ ID NOs 3-9, respectively.

The term “a variant” as described herein, refers to variants of said target RNA sequences that have substantially identical function as said target sequences, respectively. Also, said variants of said guide strand as described below have substantially the same function as said guide strands as described below. That is, said variants of said guide strands can still bind to said target RNA or said variants of said target RNA so as to further inhibit and/or reduce the transcripts encoded by said α-syn gene. Optionally, said variants of said target RNA sequences comprise at most 4 nucleotides, at most 3 nucleotides, at most 2 nucleotides or at least one nucleotide different from said target RNA sequences, respectively.

Preferably, said RNA sequence substantially complementary to said target RNA is selected from the group consisting of SEQ ID NOs 10-16 (Table 2), and variants of SEQ ID NOs 10-16, preferably consisting of SEQ ID NOs. 11, 14 and 15 and variants of SEQ ID NOs. 11, 14 and 15, more preferably consisting of SEQ ID NOs. 11 and 15 and variants of SEQ ID NOs. 11 and 15. Thus, said RNA sequence comprises one sequence selected from the group consisting of SEQ ID NO. 10 to 16 and variants of SEQ ID NO. 10 to 16.

TABLE 2
Suitable guide strands RNA sequences

| SEQ ID | | Guide RNA sequence | Length |
|---|---|---|---|
| NO. | miSNCA | (5′-3′) (miRNA sequence) | (nucleotides) |
| 10 | 02 | UCCUUGGCCUUUGAAAGUCCUU | 22 |
| 11 | 05 | UGCUCUUUGGUCUUCUCAGCCA | 22 |
| 12 | 07 | UCCAACAUUUGUCACUUGCUCU | 22 |
| 13 | 12 | UUGGUUUUCUCAGCAGCAGCCA | 22 |
| 14 | 13 | AACACCCUCUUUUGUCUUUCCU | 22 |
| 15 | 15 | UUGCCCAACUGGUCCUUUUUGA | 22 |
| 16 | 16 | UUAGGCUUCAGGUUCGUAGUCU | 22 |

As described above, said RNA sequence (i.e. guide strand) which is substantially complementary to said target RNA sequence is designed so that said RNA sequence binds to said target RNA sequence. Thereby, the transcripts, such as mRNAs, and/or proteins of the α-syn gene, can be reduced and/or knocked down.

Said variants of SEQ ID NOs 10-16 have substantially the same sequences of SEQ ID NOs 10-16, and have the same function and substantially the same binding to said target DNA as SEQ ID NOs 10-16, respectively. Optionally, said variants of SEQ ID NOs 10-16 have at least one nucleotide and at most 5 nucleotides different from SEQ ID NOs 10-16, respectively. Optionally, said variants of SEQ ID NO. 10-16 comprise at most 4 nucleotides, at most 3 nucleotides, at most 2 nucleotides or at least one nucleotide different from SEQ ID NO. 10-16, respectively. Exemplary sequences of the pri-miRNA scaffolds of the invention, comprising SEQ ID Nos 10-16, are provided in Table 3.

TABLE 3
pri-miRNA sequences

| SEQ ID | | flank -hairpin RNA sequence | Length |
|---|---|---|---|
| NO. | miSNCA | (22 nts*)-flank [5′-NNNN-3′] | (nts) |
| 17 | 02 | CUUGGGAAUGGCAAGGUCCUUGGCCUUUGAAAGUCCUU | 72 |
| | | ACUUUCAAAGGCCAAGGCUCUUGCUAUACCCAGA | |
| 18 | 05 | CUUGGGAAUGGCAAGGUGCUCUUUGGUCUUCUCAGCCA | 72 |
| | | UGAGAAGACCAAAGAGC GUCUUGCUAUACCCAGA | |
| 19 | 07 | CUUGGGAAUGGCAAGGUCCAACAUUUGUCACUUGCUCU | 72 |
| | | CAAGUGACAAAUGUUGG CUCUUGCUAUACCCAGA | |
| 20 | 12 | CUUGGGAAUGGCAAGGUUGGUUUUCUCAGCAGCAGCCA | 72 |
| | | UGCUGCUGAGAAAACCA CUCUUGCUAUACCCAGA | |
| 21 | 13 | CUUGGGAAUGGCAAGGAACACCCUCUUUUGUCUUUCCU | 72 |
| | | AAGACAAAAGAGGGUGU CUCUUGCUAUACCCAGA | |
| 22 | 15 | CUUGGGAAUGGCAAGGUUGCCCAACUGGUCCUUUUUGA | 72 |
| | | AAAGGACCAGUUGGGCA CUCUUGCUAUACCCAGA | |
| 23 | 16 | CUUGGGAAUGGCAAGGUUAGGCUUCAGGUUCGUAGUC | 72 |
| | | U UACGAACCUGAAGCCUA CUCUUGCUAUACCCAGA | |

*: nts: nucleotides

### DNA Molecules and Expression Cassettes

A second aspect of the invention relates to a nucleic acid of the invention which is a DNA molecule (“DNA molecule of the invention”). According to the present invention, a DNA molecule is preferably provided. The DNA molecule comprises a sequence corresponding to said nucleic acid sequence as described above in one of its strands.

Said DNA molecule can be useful in carrying said nucleic acid sequence as described above, and can be comprised in AAVs for being transduced in a target organ as described above.

Preferably, said DNA molecule comprises a DNA expression cassette, wherein said DNA expression cassette comprises; said nucleic acid sequence as described above; a promoter and a poly A tail; and wherein the 3′ and 5′ ends of said nucleic acid sequence are flanked by Inverted Terminal Repeats (ITRs). In other words, said DNA molecule is comprised in a DNA expression cassette, wherein in said DNA expression cassette further comprises a promoter and a poly A tail, and wherein said nucleic acid is flanked by Inverted Terminal Repeats (ITRs).

The term “DNA expression cassette” as described herein, refers to a DNA nucleic acid sequence comprising a gene or a nucleic acid sequence encoding an RNA molecule, a promoter, and a nucleic acid sequence encoding a poly A tail. Said DNA expression cassette is flanked by ITRs and is comprised in a virus vehicle and subsequently delivered to a target organ, such as the brain and/or other organs in the CNS.

The term “promoter”, as described herein, refers to a DNA sequence that is typically located at the 5′ end of transcription initiation site for driving or initiating the transcription of a linked nucleic acid sequence. In some embodiments of the invention, the promoter is a constitutive or ubiquitous promoter; a neuron-specific promoter; and/or a glial-specific promoter.

Said constitutive promoter may be selected from the group consisting of a pol II promoter, a native or engineered chicken beta-actin promoter (CBA), a CAG promoter, a PGK promoter, a CMV promoter (Such as depicted e.g. in FIG. 2 of WO2016102664, which is herein incorporated by reference).

The term “glial-specific promoter”, as described herein, refers to a promoter that may be suitably used in increasing the expression of a foreign nucleic acid and/or a gene in glial cells, such as astrocytes, oligodendrocytes or microglial cells. For expression in astrocytes, GFAP may be used. For expression in oligodendrocytes, MBP, PLP, CNP or MAG may be used. For expression in microglia, CD68 or Hexb may be used. In some preferred embodiments of the invention, the glial-specific promoter is an oligodentrocyte promoter selected from the group consisting of MBP, PLP, CNP and MAG,

Preferably, said promoter is a neuron-specific promoter. The term “neuron-specific promoter”, as described herein, refers to a promoter that may be suitably used in increasing the expression of a foreign nucleic acid and/or a gene in neuron cells, such as brain cells.

Preferably, said neuron-specific promoter is selected from the group consisting of Synapsin, Neuron-Specific Enolase (NSE), human synapsin 1, CaMKII kinase, tubulin alpha (Hioki et al. Gene Ther. 2007 June; 14 (11): 872-82), and platelet-derived growth factor-beta chain (PDGF). More preferably, said promoter comprises a dopaminergic neuron-specific promoter. Preferably said dopaminergic neuron-specific promoter is selected from TH (tyrosine hydroxylase) or Forkhead Box A2 (FOXA2).

With the use of a neuron-specific promoter in said DNA expression cassette, the expression of said nucleic acid in the CNS is induced and/or enhanced, which is preferred for reducing and/or knocking down said transcripts of α-syn gene because said transcripts of α-syn gene are expressed predominantly in the CNS such as the brain and the spinal cord and even more predominantly in the brain, and, even more predominantly, in neurons.

Other suitable promoters that can be included in the present invention are inducible and/or repressible promoters, i.e. a promoter that initiates transcription only when the host cell is exposed to a particular stimulus.

Optionally, said DNA expression cassette comprises at least two promoters including promoters as described above.

The term “poly A tail”, as described herein, refers to a long chain of adenine nucleotides that is added to a mRNA molecule for increasing the stability of the RNA molecule. Preferably, the poly A tail is the simian virus 40 polyadenylation (SV40 polyA; SEQ ID NO. 44), the Bovine Growth Hormone (BGH) polyadenylation (BGH polyA; SEQ ID NO. 45), the human Growth hormone polyadeylation (hGH polyA; SEQ ID NO. 79), or a synthetic polyadenylation. More preferably, said poly A tail is BGH poly A (SEQ ID NO. 45) or hGH polyA; SEQ ID NO. 79.

Preferably, said poly A tail comprised in said DNA expression cassette, as described above, operably links to the 3′ end of said RNA molecule, as described above.

The term “inverted terminal repeats (ITRs)”, as described herein, refers to the sequences at the 5′ and 3′ end of said DNA expression cassette, as described above, which function in cis as origins of DNA replication and as packaging signals for the virus. Said ITRs are preferably selected from a group consisting of adeno-associated virus (AAV) ITR sequences. More preferably, said ITRs sequences are both AAV1, both AAV2, both AAV5, both AAV6, both AAV7, both AAV8, or both AAV9 ITRs sequences. Also, more preferably, said ITR sequence at the 5′ end of said DNA expression cassette differs from said ITR sequence at the 3′ of said DNA expression cassette, and said ITR sequence is selected from the AAV1, AAV2, AAV5, AAV6, AAV7, AAV8, and AAV9 ITRs sequences.

Said ITRs are positioned at the left and right ends (i.e., 5′ and 3′ termini, respectively) of said nucleic acid sequence as described above. Preferably, said ITRs, as described above, are selected from a group consisting of adeno-associated virus (AAV) ITR sequences. More preferably, said ITR sequences comprise the AAV1, AAV2, AAV5, AAV6, AAV7, AAV8, or AAV9 ITR sequences. Optionally, said two ITR sequences comprise both AAV1, both AAV2, both AAV5, both AAV6, both AAV7, both AAV8, or both AAV9 ITRs sequences. Also optionally, said ITR sequence at the 5′ end of said nucleic acid sequence differs from said ITR sequence at the 3′ end of said nucleic acid sequence, wherein said ITR sequence is one selected from the AAV1, AAV2, AAV5, AAV6, AAV7, AAV8, AAV9 ITR sequences.

### AAVs

According to the present invention, an AAV vehicle (“AAV vehicle of the invention”) comprising said DNA as described above is provided.

Viral vehicles used for delivering foreign genetic material, such as said nucleic acid, or said DNA, are part of the present invention. Such viral vehicles include alphavirus, flavivirus, herpes simplex viruses (HSV), measles viruses, rhabdoviruses, retrovirus, Newcastle disease virus (NDV), poxviruses, picornavirus, lentivirus, adenoviral vectors, and preferably AAV gene delivery vehicles.

The term “AAV vehicle”, as described herein, refers to a wild-type or recombinant AAV which acts as a vehicle to carry a genetic material, such as a foreign nucleic acid, a gene of interest, a nucleic acid of interest, a vector comprising said foreign nucleic acid, a vector comprising said gene of interest, said DNA expression cassette as described above, and/or a vector comprising said DNA expression cassette as described above into a target cell, organ and/or tissue.

It was found that an AAV vehicle is a useful viral vehicle for delivery of nucleic acids or DNA expression cassettes, as described above, into a mammal. The AAV vehicle has the ability to efficiently infect dividing as well as non-dividing human cells. Moreover, said AAV vehicle has not been associated with any diseases. Hence, said AAV vehicle is useful in the present invention, and for treating and/or preventing a disease in which the α-syn gene is involved, as described below.

According to the present invention, said AAV vehicle comprises a nucleic acid comprising a nucleic acid sequence encoding a RNA, wherein a RNA sequence comprised in said RNA is substantially complementary to a target RNA sequence encoded by an α-syn gene, wherein said RNA sequence has at least 15 nucleotides, wherein said RNA includes a hairpin comprising SEQ ID NO.1, or SEQ ID NO.2, or a variant of SEQ ID NO.1 or 2. Said RNA sequence substantially complementary to said target RNA sequence is selected from a group consisting of SEQ ID NOS 10-16, and variants of SEQ ID NOs 10-16, preferably SEQ ID NOs. 11, 14 and 15 and variants of ID NOs. 11, 14 and 15, more preferably SEQ ID NOs. 11 and 15, and variants of SEQ ID NOs. 11 and 15.

Also according to the present invention, said AAV vehicle may comprise a further nucleic acid comprising a nucleic acid sequence encoding a RNA, wherein a RNA sequence comprised in said RNA is substantially complementary to a target RNA sequence encoded by an α-syn gene, wherein said RNA sequence has at least 15 nucleotides, wherein said RNA includes a hairpin comprising SEQ ID NO.1, or SEQ ID NO.2, or a variant of SEQ ID NO.1 or 2. Said RNA sequence substantially complementary to said target RNA sequence is selected from a group consisting of SEQ ID NOs 10-16, and variants of SEQ ID NOs 10-16, preferably SEQ ID NO. 11, 14 and 15 and variants of ID NO. 11, 14 and 15, more preferably SEQ ID NO. 11 and 15, and variants of SEQ ID NO. 11 and 15.

Said AAV vehicle for delivering said DNA expression cassette, as described above, is capable of modifying and/or reducing (excessive) expression levels of products encoded by the α-syn gene. Preferably said AAV vehicles are used in reducing and/or knocking down α-syn aggregates. Said α-syn aggregates typically comprise proteins encoded by said α-syn gene.

The term “decreasing” as described herein, refers to the level and/or amount of an indicated subject being reduced or lowered. The term “knocking down” as described herein, refers to the level and/or amount of an indicated subject being substantially depleted or removed.

Optionally, said AAV vehicles are used in reducing and/or knocking down transcripts encoded by mutated SNCA gene. Studies of families with a history of Parkinson's disease have resulted in the identification of a series of familial mutations leading to early-onset (A30P, E46K, A53T, G51D) or late-onset (H50Q) forms of the disease.

Preferably, said AAV vehicles are used in reducing and/or knocking down at least one isoform, including but not limited to, α-syn isoforms encoded by SEQ ID NO. 36 (SNCA140), SEQ ID NO. 76 (SNCA126), SEQ ID NO. 77 (SNCA112) or SEQ ID NO. 78 (SNCA98). More preferably, said AAV vehicles are used in reducing and/or knocking down at least one isoform which is encoded by the α-syn nucleic acid sequence comprising exon 2, 4 and/or 6.

More preferably, at least two of said RNAs which aim at reducing and/or knocking down transcripts of different target RNAs, as described above, can be combined in one AAV vehicle for further enhancing the inhibitory effect on the transcripts of α-syn gene. Therefore, the treatment and/or prevention of said diseases, as described below, is further improved.

In some embodiments of the present invention, the combined use of said RNA aiming at reducing and/or knocking down said target RNA having SEQ ID NO. 4 together with said RNA aiming at reducing and/or knocking down said target RNA having SEQ ID NO. 8 can be combined in one AAV vehicle to further enhance the inhibitory effect on the transcripts of α-syn gene as described below.

Preferably, said AAV vehicle is an AAV5, AAV8, or AAV9 vehicle. More preferably, said AAV vehicle is AAV5 or AAV9 vehicle or a hybrid thereof. The AAV vehicle of the invention may also be a AAV2/AAV5 or a AAV2/AAV9 hybrid capsid.

In some embodiments of the invention, the AAV vehicle is an AAV5 vehicle. AAV5 is useful for the present invention because the prevalence of anti-AAV5 neutralizing antibodies (Nabs) is lower than that of Nabs against other serotypes. In addition, pre-existing antibodies (Abs) or low pre-existing antibodies against AAV5 usually do not affect transduction by said AAV gene therapy vehicle, and/or expression of said nucleic acid in a target organ. Further, no cytotoxic T-cell responses against AAV5 have been reported in clinical trials.

In some embodiments of the invention, the AAV vehicle is an AAV9 vehicle. AAV9 is useful in delivering foreign nucleic acid into neuron and glial cells, including oligodendrocyte cells.

Optionally, AAV vehicles include capsids derived from AAV1, AAV2, AAV3, AAV4, AAV5, AAV6, AAV7, AAV8, AAV9, AAVrh10, AAV11, as well as variants (e.g., capsid variants with amino acid insertions, additions and substitutions, or hybrid capsids) thereof.

Preferably, the AAV vehicle of the invention comprises a capsid comprising an AAV5 and/or AAV9 and/or hybrid capsid protein sequence.

AAV capsids typically include a VP1 protein and two shorter proteins, called VP2 and VP3, that are essentially amino-terminal truncations of VP1. The three capsid proteins VP1, VP2 and VP3 are typically present in a capsid at a ratio approximating 1:1:10, respectively, although this ratio, particularly of VP3, can vary significantly and should not be considered a limitation.

Other hybrid capsids with an optimized VP1: 2:3 stoichiometry are further included in the present invention. Said optimized VP1: 2:3 stoichiometry can improve said AAV vehicles in its infectivity to a target organ and in the correct virion assembly.

AAV vehicles with capsid proteins VP1, VP2 and VP3 at a ratio approximating 1:1:10 or at an optimized VP1: 2:3 stoichiometry are useful in delivering a foreign nucleic acid sequence and/or transducing a target organ, such as an organ involved in PD, in a human subject.

As described herein and above, the AAV vehicle may be defined as “hybrid”, meaning that the viral ITRs and viral capsid are from different AAV serotypes. The viral ITRs are preferably derived from AAV2, and the capsid is preferably derived from a different one, which might be AAV5 or AAV9. Other hybrids, such as hybrids including combinations of different serotypes for capsid and ITRs, but also capsid elements from different serotypes, possibly with yet other ITRs can also be used in the present invention.

Preferably, the AAV vehicle of the invention is a gene therapy vehicle.

The term “gene therapy”, as described herein, refers to a therapy which has a steadier and/or longer-term effect than existing therapies for treating and/or preventing a disease in which the α-syn gene is involved. The preferred way to achieve a steady therapeutic effect is by a single administration of said AAV gene therapy vehicle. The stability of said therapy can be measured by common techniques known to scientists in the field. The long-term effect can be measured by the length of time the therapeutic effect lasts, and/or measured by the amount of doses and/or frequencies of injections required to maintain such therapeutic effect.

The term “treating” or “treatment”, as described herein, refers to any measures which can stop, ease, delay, slow down and/or improve the pre-symptomatic phase of said disease as described below and/or preferably at least one symptom caused by said disease, for example a neurological progressive disease. Such measures may include, but are not limited to, delaying and/or slowing down the progression of a neurological progressive disease, stopping the development of at least one symptom, easing the sickness caused by said disease, and/or improving the health condition of a patient. The term “preventing” or “prevention” as described herein, refers to any measure to stop the onset of said disease, which includes but not limit to the onset of a new symptom of said disease. The term “disease” and “disorder” can be used interchangeably in the present invention.

Said AAV gene therapy vehicle can provide a consistent effect on said expression level and/or activity level of said transcripts. Thus, in some embodiments of the present invention, said AAV gene therapy vehicle, as described above, can be used in providing consistent and/or a long-term therapeutic effects on treating and/or preventing the diseases and/or symptoms as described below. Thereby, the life quality of the patients suffering from said diseases and/or symptoms can also be improved by administering said AAV vehicle.

Said long-term effects of said AAV gene therapy vehicles can be evaluated by measuring improved outcomes of disease parameters over prolonged periods of time compared to an existing therapy for treating and/or said disease.

### Compositions

According to the present invention, a composition comprising said AAV vehicles as described above and at least one pharmaceutically acceptable excipient, is provided. Preferably, said composition comprises said AAV gene therapy vehicle.

Said composition can be in a solid form or liquid form. In some embodiments of the present invention, said composition is a formulation.

The term “additive” or “excipient” as described above and herein, refers to a substance further added into said composition, as described above, in order to give at least one function to said composition. Said functions include, but are not limited to, supplementing a property of said composition, stabilizing said composition for easy storage and/or increase of shelf-life, inhibiting side effects such as immune response, improving the transduction efficacy of said AAV vehicles to a target organ, and/or improving the bypass of the brain-blood barrier (BBB). An additive or excipient acting as a filler, without altering and/or affecting the properties of said composition, can be further included in the present invention.

Pharmaceutically acceptable excipients for administration of AAV gene delivery vehicles are well known to the skilled person and may be as simple as water for injection. They may also comprise surfactants, osmotic agents, antioxidants, etc.

Optionally, said composition further comprises an immunosuppressive compound. An immunosuppressive compound which can reduce and/or prevent an immune response induced by an injection of viral vehicles may be included in the present invention. The immunosuppressive compound may also be administered separately from the AAV composition. Such combinations are included in the invention as kits.

Optionally, a compound for improving the biodistribution of said RNA, such as said hairpin as described above, in the brain is further comprised in said composition as described above.

Optionally, said composition further comprises at least one additive selected from the group consisting of an aqueous liquid, an organic solvent, a buffer and an excipient. Optionally, the aqueous liquid is water. Also optionally, said buffer is selected from a group consisting of acetate, citrate, phosphate, tris, histidine, and 4-(2-hydroxyethyl)-1-piperazineethanesulfonic acid (HEPES). Still optionally, the organic solvent is selected from a group consisting of ethanol, methanol, and dichloromethane. Still optionally, the excipient is a salt, sugar, cholesterol or fatty acid. Still optionally, said salt, as described above, is selected from a group consisting of sodium chloride and potassium chloride. Yet optionally, said sugar, as described above, is sucrose, mannitol, trehalose, and/or dextran.

The term “target organ” as described herein refers to as an organ in which transcripts of said α-syn gene accumulates. For example, said target organ is the brain in human subjects. Other organs comprised in the CNS (i.e., brain and spinal cord) can be further included in the present invention, provided that said α-syn transcripts, such as aggregates, are present in said organs.

### Uses Thereof

The present invention provides the use of said AAV (vehicle) and/or said AAV gene therapy vehicle, as described above, as a medicament. Accordingly, the present invention also provides the use of said composition as a medicament.

The terms “AAV” and “AAV vehicle” are used interchangeably herein. Said AAV vehicle (and/or said composition comprising said AAV vehicle) as described above, can reduce and/or knock down said transcripts of an α-syn gene. Thereby, said AAV vehicles are useful in treating and/or preventing diseases caused by said transcripts of said α-syn gene, typically diseases caused by the overexpression of said transcripts encoded by said α-syn gene and/or caused by the aggregated proteins encoded by said α-syn gene.

Said transcripts are mRNA and/or proteins, and preferably mRNA. Thereby, said AAV vehicle and said composition are useful (i.e., have a therapeutic effect) in treating and/or preventing a disease in which said α-syn gene is involved. Thus, according to the present invention, said AAV vehicle and/or said composition as described above are for use as a medicament in the treatment and/or prevention of a disease in which said α-syn gene is involved.

The disease and/or symptom in which said the α-syn gene is involved, or which is caused by the transcripts encoded by said α-syn gene (e.g., by the overexpression of said transcripts), is preferably a disease caused by the overexpression of said α-syn proteins and/or by the aggregates of the α-syn proteins.

In some embodiments of the invention, said AAV vehicle and/or said AAV gene therapy vehicle and/or composition as described above are used in treating and/or preventing a disease by decreasing and/or knocking down transcripts encoded by the α-syn gene. Thus, the present invention also provides the use of said AAV vehicle and/or said AAV gene therapy vehicle and/or composition, as described above, as a medicament, wherein said medicament decreases and/or knocks down transcripts encoded by the α-syn gene.

Said disease as described above can further include diseases in which at least one single nucleotide polymorphism (SNP) of said α-syn gene is involved. For example, said diseases are caused by proteins and/or aggregates encoded at least one SNP of said α-syn gene.

Preferably, the α-syn protein (SEQ ID NO. 35) expression level is reduced by at least 30% and/or at most 70% compared to an α-syn protein expression level without administering said AAV vehicle and/or said composition. More preferably, the proteins encoded by said SNCA gene are reduced by maximally 50% compared to the endogenous α-syn protein expression level without administering said AAV vehicle and/or said composition. Complete knock down may not be preferred because of the central role of the α-syn gene.

Preferably, said AAV vehicle and/or said composition as described above, can decrease about at least 30% and/or at most 70%, more preferably maximally 50% of said transcripts as described above, compared to not administering said AAV vehicles and/or composition into a human subject. Preferably, said expression level of said transcripts is reduced by at least 30% and at most 70%, more preferably maximal 50%, compared to an expression level without administering said AAV vehicles and/or said composition. More preferably, the α-syn proteins are reduced by at least 30% and at most 70%, still more preferably maximal 50%, compared to the α-syn protein level without administering said AAV vehicles and/or said composition.

Preferably, said expression level of said transcripts is reduced by at least 30% and at most 70%, more preferably maximal 50%, compared to an expression level without administering said AAV vehicles and/or said composition into a human subject. More preferably, the α-syn proteins are reduced by at least 30% and at most 70%, still more preferably maximal 50%, compared to the α-syn protein level without administering said AAV vehicles and/or said composition into a human subject.

By using said AAV vehicles, the level of said transcripts of said α-syn gene is lowered but not completely substantially depleted. Hence, so that the diseases caused by overexpression of said transcripts and/or by the aggregates of the α-syn proteins are at least partially treated and/or prevented, and that the diseases and/or symptoms caused by the complete knock down of said transcripts are also at least partially treated and/or prevented.

In some embodiments of the present invention, said AAV vehicles and/or said composition are used in reducing and/or knocking down α-syn protein aggregates. Said α-syn protein aggregates typically comprise proteins encoded by said α-syn gene.

Preferably, said α-syn aggregates are reduced by at least 30% and/or at most 70%, more preferably maximal 50%, compared to an amount of α-syn aggregates in a patient/human subject without administering said AAV vehicles and/or said composition.

Albeit it is beneficial to decrease and/or knock down the transcripts encoded by said α-syn gene, the complete depletion (i.e. complete knock down) can result in attenuated synaptic transmission and/or neurodegeneration in the CNS, which may put patients at risk.

Thereby, said AAV vehicles and/or said composition are useful in treating and/or preventing a disease in which the α-syn gene is involved, reducing and/or preventing diseases and/or symptoms, while a substantially complete knock down is not caused, reducing and/or avoiding the risks caused by the complete depletion of said transcripts encoded by said α-syn gene.

Said AAV vehicle and/or composition as described above, can be used in treating and/or preventing said diseases caused by the formation and/or presence of oligomeric α-syn, fibrillar α-syn, aggregated α-syn, phosphorylated α-syn, Lewy bodies and/or Papp-Lantos bodies.

Preferably, said AAV vehicle and/or said composition as described above is used in decreasing and/or knocking down the amount of Lewy bodies and/or Papp-Lantos bodies.

α-syn proteins/aggregates form a majority part of Lewy and Papp-Lantos bodies (also known as Papp-Lantos inclusions). Thereby, through reducing and/or knocking down α-syn proteins, the (progression in the) amount of Lewy and/or Papp-Lantos bodies can be reduced and/or depleted. Thereby, by using said AAV vehicles and/or said composition, the accumulation of Lewy and/or Papp-Lantos bodies can be reduced to achieve the treatment and/or prevention of diseases and/or symptoms, as described below.

Thus, said AAV vehicle and/or said composition may be used as a medicament for decreasing the amount of total α-syn, oligomeric α-syn, aggregated α-syn and phosphorylated α-syn, and thus the levels of Lewy and Papp-Lantos bodies, thereby halting disease progression and/or improving disease symptoms.

Such diseases and/or symptoms include, but are not limited to, clinical symptoms of PD, LBD, MSA, neuropsychiatric symptoms, motor symptoms of PD, cognitive impairment, sleep disturbances, autonomic disturbances, and/or olfactory disturbances. Motor or movement symptoms of PD comprise rigidity of limbs, tremors, and/or impaired balance and/or coordination, or at least two of the symptoms.

Clinical symptoms of PD include, but are not limited to, rest tremor, bradykinesia, rigidity and loss of postural reflexes, secondary motor symptoms (hypomimia, dysarthria, dysphagia, sialorrhoea, micrographia, shuffling gait, festination, freezing, dystonia, and/or glabellar reflexes), and/or non-motor symptoms (such as autonomic dysfunction, cognitive/neurobehavioral abnormalities, sleep disorders, sensory abnormalities such as anosmia, paresthesias and/or pain).

Clinical symptoms of LBD include, but are not limited to, movement disorders typical of PD, such as rigidity of limbs, tremors, and/or impaired balance and/or coordination, intellectual decline, visual hallucinations, poor regulation of body functions (autonomic nervous system), sudden changes in attention and mood, cognitive problems, sleep difficulties, fluctuating attention, and depression and apathy.

Clinical symptoms of MSA include, but are not limited to, movement disorders typical of PD, sexual dysfunction, urinary disfunction, REM sleep behavior disorder, orthostatic hypotension, stridor, parkinsonism, cerebellar features, multidomain autonomic failure, pyramidal signs and/or frontal executive dysfunction.

Thus, the AAV vehicle or composition for use as a medicament is used for treating and/or preventing clinical symptoms of PD, LBD, MSA, neuropshychiatric symptoms, motor symptoms of PD, cognitive impairment, sleep disturbances, autonomic disturbances, and/or olfactory disturbances. Preferably, said AAV vehicle and/or composition as described above, is used in the treatment and/or prevention of PD, MSA and/or LBD. Preferably, said disease is PD and/or MSA.

Overexpression of said α-syn gene, aggregation of said α-syn proteins, and/or the formation and/or presence of Lewy bodies are indicators of a patient suffered from PD. By using said AAV vehicle and/or composition as described, the transcripts of said α-syn gene can be reduced and/or knocked down. Thereby, said AAV vehicle and/or composition is useful in treating and/or preventing PD.

Preferably, said AAV vehicle and/or composition is used in treating and/or preventing PD patients in their pre-symptomatic phase or symptomatic phase.

The term “pre-symptomatic phase” as described herein, refers to a phase in a neuron progressive disease such as PD before the onset of clinical disease.

The term “symptomatic phase” as described herein, refers to a phase in a neuron progressive disease such as PD after clinical diagnosis of said disease.

PD patients usually notice that they have PD when at least one of said symptoms as described above appears. However, it may be too late to treat and/or prevent the disease progression because a large portion of neurons are lost in a pre-symptomatic phase. Therefore, it is useful to have a therapy such as the use of said composition as described above, in treating and/or preventing the disease progression before at least one symptom of PD, such as motor symptoms, shows.

As described above, said AAV vehicles and/or said composition can decrease the α-syn protein level, and thereby said AAV vehicles and/or said composition are useful in treating and/or preventing at least one PD symptom. Said symptom can be selected from a group consisting of depression, sleep disturbances, pain and fatigue at earlier stages of the disease, and anxiety, dementia and cognitive dysfunction at later disease stages.

Similarly, deposits of Lewy bodies may cause a form of dementia called Lewy body dementia, or LBD. Indeed, LBD causes some or all of the motor symptoms of Parkinson's. Thus, said AAV vehicles and/or said composition may also prove useful in treating and/or preventing at least one PD symptom.

Additionally, overexpression of said α-syn gene and/or the aggregated proteins encoded by said α-syn gene can increase the risk of MSA, a progressive brain disorder that affects and/or hinders movement and balance and/or disrupts the function of the autonomic nervous system. The disease was first known as Shy-Drager Syndrome. Currently, it is believed that MSA is “sporadic” meaning that there are no established genetic or environmental factors that cause the disease.

Although many clinical symptoms are also present in those with Parkinson's disease, patients with MSA typically show symptom onset at a younger age, with the average onset in the early 50s. Many patients are diagnosed with Parkinson's disease first, but over time, the extent, severity, and type of symptoms change, making a diagnosis of MSA more likely.

Important differences distinguish the symptoms and course of MSA from Parkinson's disease. Notably, MSA affects several areas of the brain, including the cerebellum, the brain's balance and coordination centers, and the autonomic nervous system, as mentioned above. Further, while Parkinson's disease affects the dopamine-producing neurons of a motor-controlling portion of the brain known as the nigro-striatal area, MSA affects both neurons and glial cells.

In MSA, hyperphosphorylated α-syn is found in Papp-Lantos inclusions (or GCIs). Thereby, the AAV vehicles and/or composition of the invention can also be useful in decreasing and/or inhibiting the amount of Papp-Lantos inclusions and in treating and/or preventing MSA.

Other diseases, such as CNS diseases may be treated and/or prevented by the similar approach of using an AAV vehicle and/or a composition comprising an AAV vehicle, wherein said diseases are caused by overexpression of a gene while a complete knock out of the transcripts of said gene is less desired.

Further, in different phases (i.e. stages) of progressive neurological disorders caused by the accumulation of the transcripts of the α-syn gene, a patient may develop different symptoms and/or different sickness levels. Said AAV vehicle and/or said composition provide a solution for treating and/or preventing said different symptoms and/or different sickness levels, without the constant modification of the therapy regimens.

### Methods

According to the present invention, a method for producing said AAV vehicle as described above, is provided.

Optionally, said AAV vehicles can be produced by using mammalian cells. Optionally, said AAV vehicles can be produced by using insect cells, preferably baculovirus. Suitable methods of production of AAV gene therapy vehicles comprising such DNA expression cassette, as described above, are described in WO2007/046703, WO2007/148971, WO2009/014445, WO2009/104964, WO2011/122950, WO2013/036118, which are incorporated herein in its entirety and particularly referred to for their methods of production. Optionally, said composition further comprises said immunosuppressive compound.

According to the present invention, a method for producing said composition as described above, is provided.

### Kits

For the purpose of treating and/or preventing the diseases or disorders as described above, said AAV vehicles as described above and at least one additive, as described above, can be combined into a kit. Said kit can optionally include means for retaining and/or containing said AAV vehicles and at least one said additive.

In some embodiments of the invention, as explained above, the kit comprises the AAV vehicles of the invention and an immunosuppressive compound as described above. Medical practitioners and patients can readily follow the labels and/or the instructions to apply said AAV vehicles as described above on a human subject.

It is understood that kits comprising compositions comprising the AAV vehicles of the invention and at least one pharmaceutically acceptable excipient are also provided. Thus, optionally, said kit further comprises at least one additive selected from the group consisting of an aqueous liquid, an organic solvent, a buffer and an excipient. Optionally, the aqueous liquid is water. Also optionally, said buffer is selected from a group consisting of acetate, citrate, phosphate, tris, histidine, and 4-(2-hydroxyethyl)-1-piperazineethanesulfonic acid (HEPES). Still optionally, the organic solvent is selected from a group consisting of ethanol, methanol, and dichloromethane. Still more, the excipient is a salt, sugar, cholesterol or fatty acid. Still optionally, said salt, as described above, is selected from a group consisting of sodium chloride, potassium chloride. Yet optionally, said sugar, as described above, is sucrose, mannitol, trehalose, and/or dextran.

### DRAWINGS OF THE INVENTION

FIG. 1. Alternatively spliced variants of SNCA mRNA (SNCA140, SNCA126, SNCA112, SNCA98), and regions within targeted by the miSNCA candidate sequences (candidates 2, 5, 7, 12, 13, 15 and 16).

FIG. 2. Vector maps for (A) the original expression cassette with a miR451 backbone only and (B) the improved expression cassette with miR144 included in the backbone.

FIG. 3. Dual Luciferase assays titration of miSNCA candidates

FIG. 4. Dual Luciferase assays: tTitration of the original and improved miSNCA5 and miSNCA15.

FIG. 5. Dose-dependent endogenous α-syn lowering in HEK293T cells at both mRNA and protein levels. A significant correlation between SNCA mRNA and α-syn protein levels was observed.

FIG. 6. Expression levels of miSNCA5 (A) and miSNCA15 (B) relative to the endogenous miRNAs.

FIG. 7. miRNA processing of miSNCA5 and miSNCA15.

FIG. 8 pVD1502 plasmid ap of plasmid.

FIG. 9. Route of administration study in wild-type (wt) rats. (A) AAV5-GFP vDNA levels; (B) AAV5-GFP mRNA expression relative to GAPDH as housekeeping gene.

FIG. 10. Mechanism of action study in a-syn KI rats. (A) vDNA levels; (B) miSNCA5 levels; (C) miSNCA15 levels; (D) SNCA mRNA lowering in striatum.

FIG. 11. Rescue of motor phenotype in C. elegans PD model by miSNCA candidates.

FIG. 12. Processing of miRNAs extracted from rat brain tissue from in vivo study #2 and from pooled samples of group 4 from this study.

FIG. 13. vDNA levels in striatum in in vivo study 3 (AAV1/2-hA53T-aSyn rat model of Parkinson's disease). vDNA levels were comparable in all groups receiving the same dose of the AAV5-unrelated miR or AAV5-miSNCA treatments

FIG. 14. miSNCA levels in striatum in in vivo study 3 (AAV1/2-hA53T-aSyn rat model of Parkinson's disease); (A) miSNCA5 levels were high in the groups injected with AAV5-miSNCA5; (B) miSNCA15 levels were high in the groups injected with AAV5-miSNCA15.

FIG. 15. Human SNCA mRNA levels in striatum in in vivo study 3 (AAV1/2-hA53T-aSyn rat model of Parkinson's disease); human SNCA mRNA levels were the highest in the AAV1/2-hA53T-aSyn groups co- or sequentially injected with AAV5-unrelated miR. In the AAV1/2-hA53T-aSyn groups co- or sequentially injected with AAV5-miSNCA5 or AAV5-miSNCA15, levels of human SNCA mRNA levels were significantly lower.

FIG. 16. Human α-syn protein levels in striatum in in vivo study 3 (AAV1/2-Ha53T-aSyn rat model of Parkinson's disease); human α-syn protein levels were the highest in the AAV1/2-Ha53T-aSyn groups co- or sequentially injected with AAV5-unrelated miR. In the AAV1/2-Ha53T-aSyn groups co- or sequentially injected with AAV5-miSNCA5 or AAV5-miSNCA15, levels of human α-syn protein levels were significantly lower.

FIG. 17. Dopamine transporter levels (assessed by ([125I]-RTI-121 autoradiography) in striatum in in vivo study 3 (AAV1/2-hA53T-aSyn rat model of Parkinson's disease); dopamine transporter levels were significantly reduced in the AAV1/2-hA53T-aSyn group sequentially injected with AAV5-unrelated miR (similarly to what is observed in PD patients). Sequential injection of AAV5-miSNCA5 or AAV5-miSNCA15 rescued the dopamine transporter loss.

FIG. 18. Dopamine and dopamine metabolite levels (assessed by LC/MS) in striatum in in vivo study 3 (AAV1/2-hA53T-aSyn rat model of Parkinson's disease); (A) dopamine levels were significantly reduced in the ipsilateral striatum of the AAV1/2-hA53T-aSyn group sequentially injected with AAV5-unrelated miR (similarly to what is observed in PD patients). Sequential injection of AAV5-miSNCA5 or AAV5-miSNCA15 rescued the dopamine loss. (B) the ratio between dopamine metabolites and dopamine was significantly increased in the ipsilateral striatum of the AAV1/2-hA53T-aSyn group sequentially injected with AAV5-unrelated miR, indicating deficit in dopamine turnover (similarly to what is observed in PD patients). Sequential injection of AAV5-miSNCA5 or AAV5-miSNCA15 rescued this deficit in dopamine turnover.

FIG. 19. Motor behavior test (cylinder test, assessing paw use asymmetry) in in vivo study 3 (AAV1/2-hA53T-aSyn rat model of Parkinson's disease). (A) At baseline (before any injections) there was no paw use asymmetry in any of the treatment groups; (B) Underuse of the contralateral paw was observed at day 56 post-treatment in the AAV1/2-hA53T-aSyn group sequentially injected with AAV5-unrelated miR (similarly to what is observed in PD patients). Sequential injection of AAV5-miSNCA5 or AAV5-miSNCA15 rescued this motor deficit.

FIG. 20. (A) TH, (B) human α-syn and (C) α-syn positive TH neurons in the substantia nigra of in vivo study 3 (AAV1/2-hA53T-aSyn rat model of Parkinson's disease), assessed by immunohistochemistry; only sequentially injected groups were assessed. (A) Substantia nigra TH positive cells were significantly reduced in AAV1/2-hA53T-aSyn animals sequentially injected with AAV5-unrelated miR, with respect to AAV1/2-empty vector animals sequentially injected with AAV5-unrelated miR (similarly to what is observed in PD patients). Sequential injection of AAV5-miSNCA5 or AAV5-miSNCA15 rescued the TH neuron loss. (B) Substantia nigra α-syn positive cells were reduced by AAV5-miSNCA5 or AAV5-miSNCA15, confirming target engagement. (C) TH positive cells in substantia nigra showed significantly less expression of a-syn in the AAV5-miSNCA5 or AAV5-miSNCA15 treated groups.

FIG. 21. Reduction of SNCA mRNA and α-syn protein expression in C. elegans PD model by miSNCA candidates, assessed by RT-qPCR and western blot, respectively. (A) SNCA mRNA levels, treatment at L1 stage and measured at day 1; (B) SNCA mRNA levels, treatment at L4 stage and measured at days 1, 4, 8 and 11; (C) SNCA mRNA levels, treatment at day 1 and measured at days 1, 4, 8 and 11; (D) α-syn protein levels, treatment at L1 stage and measured at day 1; (B) α-syn protein levels, treatment at L4 stage and measured at days 1, 4, 8 and 11; (C) α-syn protein levels, treatment at day 1 and measured at days 1, 4, 8 and 11. Both SNCA mRNA and α-syn protein levels were reduced by miSNCA candidates, with respect to EV treated groups.

FIG. 22. Motor phenotype rescue in C. elegans PD model by miSNCA candidates, after treatment at (A) L1, (B) L4 or (C) day 1 stage.

FIG. 23. Small RNA sequencing results of C. elegans samples that were treated with full length SNCA RNAi, miSNCA5 and miSNCA15 miRNAs at L1 stage and collected at Day 1 and Day 4 of their adulthood after treatment. The (A) miSNCA5 and (B) miSNCA15 are correctly processed and can be found in relevant samples. (C) The miSNCA5 and miSNCA15 sequences, as well as all the other designed miSNCAs (miSNCA2, miSNCA7, miSNCA12, miSNCA13, miSNCA16), within the full length SNCA treated C. elegans samples.

### EXAMPLES OF THE PRESENT INVENTION

### Materials and Methods

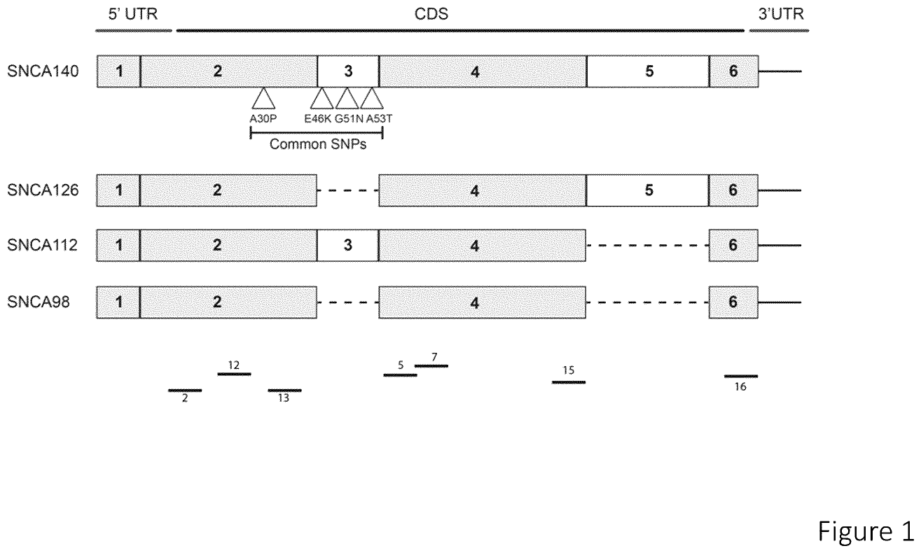

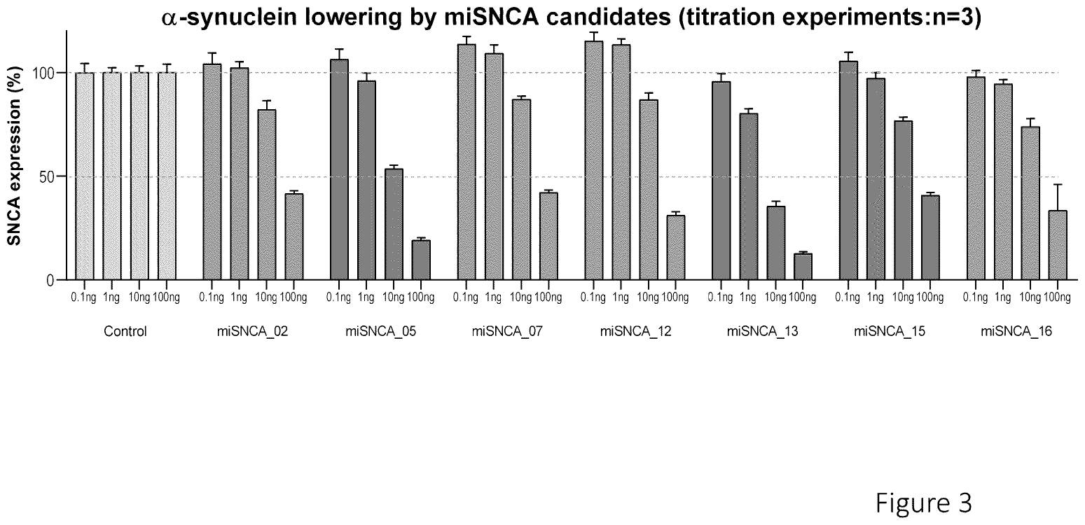

SNCA miRNA Guide Strand Design.
miSNCAs (miRNA guide strands) were designed to target common RNA sequences of the most common SNCA mRNA variant; SNCA140, SNCA126, SNCA112 and SNCA98. miRNAs were designed in regions that are common with all of the major mRNA variants of SNCA (FIG. 1) (McLean et al. 2012 Mol and Cell Neuroscience 49 (2) 230-239). The target regions of the SNCA mRNA sequences are a portion of exon 2, exon 4 and exon 6. The most common SNP outside of these exons (A30P), was avoided to be included in the guide RNAs. Each of the conserved sequences was used to generate a number of different guide strands having 22 nucleotides (nts). Seventeen guides targeting SNCA were designed and incorporated into miR451 scaffold: miSNCA2 (SEQ ID NO. 24), miSNCA5 (SEQ ID NO. 25), miSNCA7 (SEQ ID NO. 26), miSNCA12 (SEQ ID NO. 27), miSNCA13 (SEQ ID NO. 28), miSNCA15 (SEQ ID NO. 29), miSNCA16 (SEQ ID NO. 30), miSNCA1 (SEQ ID NO. 80), miSNCA3 (SEQ ID NO. 81), miSNCA4 (SEQ ID NO. 82), miSNCA6 (SEQ ID NO. 83), miSNCA9 (SEQ ID NO. 84), miSNCA10 (SEQ ID NO. 85), miSNCA11 (SEQ ID NO. 86), miSNCA14 (SEQ ID NO. 87), miSNCA18 (SEQ ID NO. 88), miSNCA19 (SEQ ID NO. 89). These constructs were only containing miR451 as the backbone not the miR144 helper. These were tested in vitro for their efficacy to lower the expression of concatenated SNCA reporter gene (SEQ ID NO. 34) in Dual luciferase reporter plasmid using Dual Luciferase assays. Out of these seventeen miRNAs, seven of them (pri-miRNA of miSNCA2, miSNCA5, miSNCA7, miSNCA12, miSNCA13, miSNCA15 and miSNCA16; SEQ ID NO. 17-23) that were coded in the ITR containing vectors (SEQ ID NO. 24-30) showed potential to lower the SNCA mRNA levels in a dose-dependent manner (FIG. 3).

The miSNCA guides were selected based on the following criteria: the miRNA guide sequences should not include a stretch of >4 G, >4 C, >5 A and >5 T nt; they should have a GC content between 30% and 70%; <4000 predicted off-target genes of the miRNA seed sequence for exon 1a targeting guides and <5000 predicted off-target genes of the miRNA seed sequence for intron 1 targeting guides by using siSPOTR analysis (https://sispotr.icts.uiowa.edu./sispotr/tools/lookup/evaluate.html); and pre-miRNA sequence folding energy between −44 and −55 kcal/mole. To generate a negative control scramble guide was designed for in vitro testing and named miSCR (SEQ ID NO. 90).

The selected miSNCA guides meet the following criteria: conservation with monkey SNCA gene sequence (Macaca mulatta, NCBI accession number NC_041768.1); the miRNA guide sequence does not include a stretch of >4 G or >4 C nt; it has a GC content between 20% and 70%; a GC seed content between 40% and 70%; pre-miRNA sequence folding energy between −45 and −55 kcal/mole; and no matching with endogenous miRNA seeds.

Guide sequences were incorporated into human pri-miRNA miR-451 scaffold sequences and the mFold program (http://unafold.rna.albany.edu/?q=mfold) was used with standard settings to determine whether the candidates were folded into the secondary structures.

The original SNCA scaffold (scaffold 1) consists of only one miR451 as scaffold. The improved SNCA scaffold (scaffold 2) consists of the miR-144 hairpin and one mir-451 downstream scaffold. Scaffold 2 is the improved version of the original constructs and contains miR144, which is a helper for the processing of the miSNCAs. Placement of the miR144 hairpin is always at the 5′ end of (most compared to) the miR451 hairpin sequence. Seven SNCA constructs (scaffold 1) (SEQ ID NOs. 37-43) were generated to target the SNCA mRNAs and two improved SNCA constructs (scaffold 2) were generated to target different parts of the SNCA mRNA (SEQ ID NO. 91 & 92).

### Dual Reporter Luciferase Assay

For Dual Luciferase assays and endogenous α-syn lowering, HEK293T cells were used. For Dual Luciferase assays the HEK293T cells (1×10⁵ cells/well) were plated into 24-well tissue culture-treated plates in triplicates. The cells were co-transfected with reporter plasmid (SEQ ID NO. 33) carrying concatenated SNCA reporter sequence (SEQ ID NO. 34) (10 ng) and varying amounts (0.1-1-10-100 ng) of plasmid carrying miSNCA candidates using Lipofectamine 3000 (Thermo Fisher Scientific). The cells were then collected after two days of transfection and the cell samples were analyzed for Renilla Luciferase and Firefly Luciferase activity using the Dual Luciferase assay kit from Promega. The assays were performed in GloMax Luminescence reader. α-syn lowering was measured as a decrease in the RL/FL activity ratio. The experiments were repeated an average of three times.

### Transfections and Endogenous α-Syn Lowering

To evaluate endogenous α-syn lowering by miSNCA candidates, HEK293T cells were used. For these assays the HEK293T cells (5×10⁵ cells/well) were plated into 6-well tissue culture-treated plates in triplicates. The cells were transfected with varying amounts (50-200-1000 ng) of plasmid carrying miSNCA candidates using Lipofectamine 3000 (Thermo fisher Scientific). The cells were then collected two days after the transfections. The cell samples were analyzed for the mRNA levels of SNCA and α-syn protein levels. The experiments were repeated an average of three times.

### DNA Constructs for Baculovirus Seed Generation

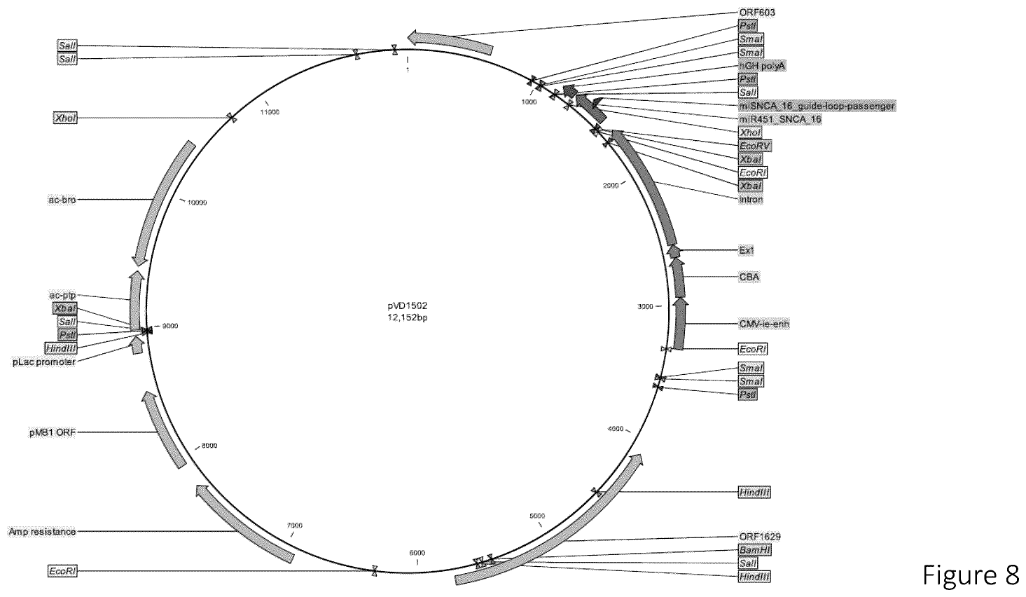

The expression cassettes carrying the different miSNCA constructs were subcloned into ITRs containing plasmids generating pVD1496 (SEQ ID NO. 37), pVD1497 (SEQ ID NO. 38), pVD1498 (SEQ ID NO. 39), pVD1499 (SEQ ID NO. 40), pVD1500 (SEQ ID NO. 41), pVD1501 (SEQ ID NO. 42) and pVD1502 (SEQ ID NO. 43; FIG. 8).

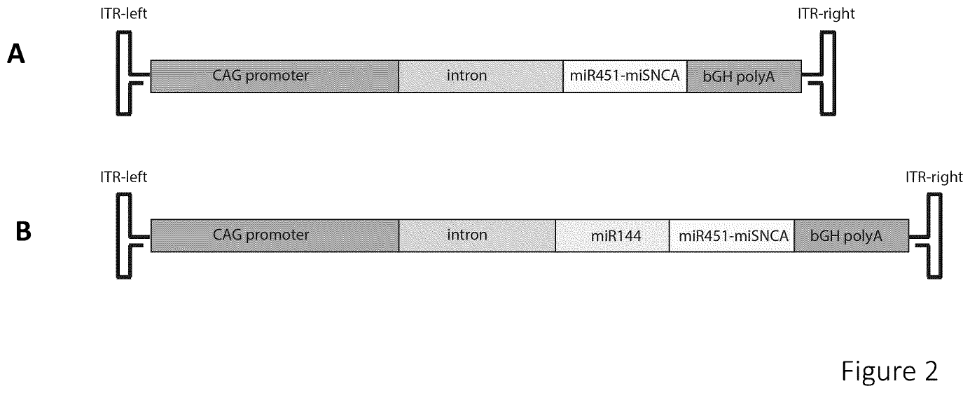

All these pVD plasmids carry a CAG promoter, and an intron necessary for the promoter activity, followed by the miSNCA constructs in miR451 backbone (SEQ ID NOs. 24-30) and the bGH polyA sequence (FIG. 2A).

Improved construct versions of miSNCA5 and miSNCA15 were also created by incorporating miSNCA5 or miSNCA15 guide sequences into the miR451 that is downstream of miR144 helper miRNA (scaffold containing miR 144 and miR451; FIG. 2B), thus creating miR144-miSNCA5 (SEQ ID NO. 31) and miR144-miSNCA15 (SEQ ID NO. 32) These expression cassettes were subcloned into pVD1587 (SEQ ID NO. 91) and pVD1588 (SEQ ID NO. 92), containing ITR regions for AAV5 packaging.

### AAV5 Vectors

Recombinant AAV5 harboring the expression cassettes were produced by infecting SF+ insect cells (Protein Sciences Corporation, Meriden, Connecticut, USA) with two baculoviruses encoding Rep, Cap and Transgene. Following standard protein purification procedures on a fast protein liquid chromatography system (AKTA Explorer, GE 30 Healthcare) using AVB sepharose (GE Healthcare), the titer of the purified AAV was determined using QPCR.

### In Vitro Models and Transduction Assays

To measure the effects of AAV5-miSNCA on human α-syn mRNA and protein levels, patient-derived iPSC-derived Dopaminergic neurons (DA neurons) were used. IPSC cell lines (Table 4) were obtained from NINDS RUCDR repository.

TABLE 4
iPSC cell lines and controls

| Short name | Subject ID | Cell Line ID | Gene affected | Genotype |
|---|---|---|---|---|
| A53T | NDS00188 | ND50050 | SNCA | ALA53THR |
| Isogenic control | NDS00188 | ND50085 | SNCA | Isogenic (edited) |
| Edited | NDS00188 | ND50086 | SNCA | Heterozygous (edited) |
| (heterozygous) | | | | |
| SNCA3X | NDS00201 | ND50040 | SNCA | SNCA triplication |
| Control | NDS00159 | ND41865 | none | Non-patient control |

The iPSC cells were differentiated into DA neurons using PSC Dopaminergic neuron Differentiation kit from Thermo Fisher Scientific.

The above mentioned in vitro cell models were transduced using baculovirus-produced AAV5-miSNCA candidates at various multiplicity of infection (MOI) of the virus. The cells were plated into PDL-Laminin, or PLO-Laminin coated 6-well plates at 5×10⁵ cells/well. After 3-4 days of passaging, the cells were transduced at MOI of 10⁴, 10⁵, 10⁶ and 10⁷/cell. The cells were then collected at 7-15 days after the transduction. The cell samples were used for RNA and DNA isolation to determine vector DNA levels, miSNCA expression, SNCA mRNA and α-syn protein expression.

### RNA Isolation and Small RNA Sequencing Using Next-Generation Sequencing (NGS)

a. From HEK-Produced AAV5-miSNCA Candidates

The RNA was isolated from the AAV5-miSNCA (HEK-produced) transduced-DA neurons (with MOI of 10⁶/cell) using Allprep DNA/RNA Micro kit (Qiagen). The RNA integrity was determined using Bioanalyzer and RNA quantified using Nanodrop. The samples were then sent for small RNA sequencing to GenomeScan BV (Leiden, Netherlands). Small RNA sequencing was performed by GenomeScan using the NebNext small RNA library prep method including BluePippin size selection of the final library combined with Illumina NovaSeq6000 PE150 sequencing. The data was analyzed using CLC Genomics Suit (Qiagen). Expression values of the miSNCA candidates were expressed as the RNA counts of miSNCA candidates versus the whole annotated miRNA sequence counts. The processing of the miSNCA candidates was analyzed by aligning miSNCA raw pri-miRNA sequences against the sequenced RNA molecules. Various sizes of miSNCA molecules and their counts were obtained.

b. From Baculovirus-Produced AAV5-miSNCA Candidates

The RNA is isolated from the AAV5-miSNCA candidate (Baculovirus produced) transduced cells (DA neurons, forebrain neurons and/or LUHMES derived DA neurons) using Zymogen RNA isolation kit. The RNA quality is tested using Bioanalyzer and quantified using Nanodrop. Then the samples are sent for small RNA sequencing to GenomeScan BV (Leiden, Netherlands) using next generation sequencing methods. The data is analyzed using CLC Genomics Suit (Qiagen), to extract information about the expression values of the miSNCA candidates and also to find out the processing of the miSNCA candidates that were expressed from the Baculovirus produced AAV5-miSNCA candidates.

RNA was isolated from the rat brain striatum samples from the in vivo study #2, group #4 (described below). These animals were injected in their striatum with both AAV5-miSNCA5 and AAV5-miSNCA15, the processed sequences of which were found in the sequencing analysis of the samples. RNA was isolated using AllPrep DNA/RNA isolation kit. The RNA quality was tested using Bioanalyzer and quantified using Nanodrop. Then the samples were sent for small RNA sequencing to GenomeScan BV (Leiden, Netherlands) using next generation sequencing methods. The data was analyzed using CLC Genomics Suit (Qiagen), to evaluate the processing of the miSNCA candidates that were expressed from the Baculovirus produced AAV5-miSNCA candidates.

### Small RNA Sequencing (NGS) Data Analysis

Data analysis was carried out using CLC Genomics Workbench 10 suit. The trimmed small RNA sequence reads were counted and annotated using miRbase database. The miSNCA molecules are annotated by aligning the pri-miRNA sequence against these small RNA library. The expression values of the miSNCA candidates were expressed as the number of counts of miSNCA candidate counts versus the whole annotated small RNA counts. The most expressed miSNCA molecules were analyzed by looking at the relative counts of the various sizes of the miSNCA, aligning the pre-miSNCA to the small RNA library and using the RNA counts obtained from there.

Vector DNA Isolation and Quantification from Cells and Animal Tissues

DNA extraction was performed using AllPrep DNA/RNA Mini Kit (Qiagen) following manufacturer's instructions. Vector genome copies were quantified by using TaqMan qPCR assay (Thermo Fisher scientific) with primers against the poly A region of the vector. The quantification (GC/ug DNA) was done using linearized pVD plasmid and making a standard curve with varying amounts of this linearized plasmid. The standard curve created this way was used to calculate the vector DNA copy number from the DNA isolated from cells transduced with AAV5-miSNCAs.

RNA and Protein Isolation from Transfected HEK Cells and Quantification of mSNCA and α-Syn Protein Levels

For the RNA isolation, the Direct-zol™ RNA Miniprep (Catalog no. R2050) was used. TRIzol was applied to the snap frozen cell pellets to lyse them. The cDNA syntheses were performed using the Maxima First Strand cDNA Synthesis Kit (Thermo Fisher Scientific) for RT-qPCR.

For protein isolations, RIPA buffer (Sigma) containing PhosSTOP phosphatase inhibitor (Roche) and EDTA-free protease inhibitor (Roche) was used. For extraction of the protein the buffer was added into the cell pellet and the cells were agitated at 4 C, 400 rpm for 30 minutes. The cell extract was then centrifuged at top speed. The clarified supernatant was used for α-syn and total protein measurement i.e. HTRF and BCA assays.

For the detection of mSNCA levels, SYBR Green based RT-qPCR assay was used using a set of primers designed against SNCA (Table 5). The results were displayed as fold change using the ΔΔCycle threshold (ΔΔCt) of the treated sample against the untreated sample normalized to the mean expression of household genes; UBE, CYC1 and ACTB (Table 5).

TABLE 5
Primer sequences for RT-qPCR of HEK cells.

| Primer | Type of | | |
|---|---|---|---|
| for | gene | Forward primer | Reverse primer |
| SNCA1 | transgene | AAATGTTGGAGGAGCAGTGG | TCCAGAATTCCTTCCTGTGG |
| | | (SEQ ID NO. 48) | (SEQ ID NO. 49) |
| UBE | House- | TGCCTGAGATTGCTCGGATCT | TCGCATACTTCTGAGTCCAT |
| | keeping | (SEQ ID NO. 50) | TC |
| | gene | | (SEQ ID NO. 51) |
| CYC1 | House- | AGCCTACAAGAAAGTTTGCCT | TCTTCTTCCGGTAGTGGATC |
| | keeping | AT | TT |
| | gene | (SEQ ID NO. 52) | (SEQ ID NO. 53) |
| ACTB | House- | GTCTTCCCCTCCATCGTG | TCTTGCTCTGGGCCTCGT |
| | keeping | (SEQ ID NO. 54) | (SEQ ID NO. 55) |
| | gene | | |

For detection of α-syn protein levels, the Total α-syn HTRF kit (Cisbio) was used. The HTRF measurements were then normalized by total protein added to the HTRF assay. Total protein measurements were done using a bicinchoninic acid assay (BCA Protein assay kit; Pierce™). The HTRF results were given as the HTRF ratio/μg total protein.

RNA Isolation and Quantification of miSNCA Candidates, GFP mRNA and SNCA mRNA from Animal Tissues

Tissue was homogenized using the Tissue Lyser system (Qiagen) and AllPrep DNA/RNA Mini kit (Qiagen) following the manufacturer's instructions. DNA and RNA quantity and integrity were determined by Nanodrop and Bioanalyzer.

For miSNCA expression, the following protocol was used: total RNA was isolated using AllPrep DNA/RNA Micro kit (Qiagen). RT-qPCR using a Taqman stem-loop-miRNA assay (Thermo Fisher) designed for detecting 23nts miSNCA5 and 22nts miSNCA15 was used. The expression levels were expressed as miRNA molecules/ug total RNA.

The assay ID for these Taqman assays (Thermo Fisher) are: for miSNCA5_23nt, CTNKRV7; and for miSNCA15_22nt, CTTZ9KY. For mRNA expression, total RNA was isolated using AllPrep DNA/RNA Micro kit (Qiagen).

Two different RT-qPCR assays were used to measure SNCA and GFP mRNA expression:
1. A SYBR Green based RT-qPCR assay was used to measure SNCA or GFP mRNA expression. The genes that were used as house-keeping genes are; ACTB, B2M, GAPDH, HPRT. Primer sequences are shown in Table 6.
2. Taqman assays for SNCA mRNA. The primer and probe sequences that were designed for SNCA mRNA expression and Taqman IDs for the ready-to-use Taqman assays for the house-keeping genes is given in Table 7.

TABLE 6
Primer sequences for SYBR Green-based RT-qPCR from animal tissues.

| Primer | Type of | | |
|---|---|---|---|
| for | gene | Forward primer | Reverse primer |
| SNCA1 | transgene | AAATGTTGGAGGAGCAGTG | TCCAGAATTCCTTCCTGTGG |
| | | G | (SEQ ID NO. 49) |
| | | (SEQ ID NO. 48) | |
| SNCA2 | transgene | ATGTTGGAGGAGCAGTGGT | TGTCAGGATCCACAGGCATA |
| | | G (SEQ ID NO. 56) | (SEQ ID NO. 57) |
| GFP | transgene | AGCAAAGACCCCAAC | GCGGCGGTCACGAAC TC |
| | | GAGAA (SEQ ID NO. 58) | (SEQ ID NO. 59) |
| B2M | House- | GCCATCCACCGGAGAATG | GGTGGAACTGAGACACGTAGC |
| | keeping | (SEQ ID NO. 60) | A (SEQ ID NO. 61) |
| | gene | | |
| GAPDH | House- | TGCCCCCATGTTTGTGATG | GCTGACAATCTTGAGGGAGTTG |
| | keeping | (SEQ ID NO. 62) | T (SEQ ID NO. 63) |
| | gene | | |
| ACTB | House- | AGCGTGGCTACAGCTTCAC | AAGTCTAGGGCAACATAGCAC |
| | keeping | C (SEQ ID NO. 64) | AGC (SEQ ID NO. 65) |
| | gene | | |
| HPRT | House- | GCGAAAGTGGAAAAGCCA | GCCACATCAACAGGACTCTTGT |
| | keeping | AGT (SEQ ID NO. 65) | A (SEQ ID NO. 67) |
| | gene | | |

TABLE 7
Primer sequences for Taqman RT-qPCR from animal tissues.

| Primer | Type of | | | |
|---|---|---|---|---|
| for | gene | Forward primer | Reverse primer | Probe sequence |
| SNCA1 | transgene | AAATGTTGGAGGA | TCCAGAATTCCTTC | AGCAGGGAGCATTGC |
| | | GCAGTGG | CTGTGG | AGCAGC (SEQ ID NO. |
| | | (SEQ ID NO. 48) | (SEQ ID NO. 49) | 68) |
| SNCA2 | transgene | ATGTTGGAGGAGC | TGTCAGGATCCAC | CAGCAGCCACTGGCT |
| | | AGTGGTG (SEQ ID | AGGCATA (SEQ ID | TTGTCAAA (SEQ ID |
| | | NO. 56) | NO. 57) | NO. 69) |

LC-MS/MS from Animal Tissues

Striatal tissue samples were sent on dry-ice to the Vanderbilt Neurochemistry Core Facility (Nashville, TN, USA) for determination of catecholamine levels and data sent back to Atuka blinded for analysis.

Tissue Extraction. Brain sections were homogenized, using a tissue dismembrator, in 100-750 μl of 0.1M TCA containing 10-2 M sodium acetate, 10-4M EDTA, and 7.5% methanol (pH 3.8). 10 μl of homogenate was removed for measurement of protein concentration. The samples were then spun in a microcentrifuge at 10,000 g for 20 minutes at 4° C. Supernatant was transferred to a new microcentrifuge tube for biogenic amine analysis.

Biogenic Amine Analysis. Dopamine, HVA, and DOPAC levels were determined by a highly sensitive and specific liquid chromatography/mass spectrometry (LC-MS/MS) methodology following derivatization of analytes with benzoyl chloride (BZC). 5 μl of supernatant was treated with 10 μl each of 500 mM NaCO₃ (aq) and 2% BZC in acetonitrile. After four minutes, the reaction was stopped by the addition of 10 μl internal standard solution (in 20% acetonitrile containing 3% sulfuric acid) containing 200 μg of each ¹³C₆-derivatized dopamine-d₄, HVA, and DOPAC. Liquid Chromatography was performed on a 2.0×50 mm, 1.7 μm particle Acquity BEH C18 column (Waters Corporation, Milford, MA, USA) using a Waters Acquity UPLC. Mobile phase A was 0.15% aqueous formic acid and mobile phase B was acetonitrile. Samples were separated by a gradient of 98-5% of mobile phase A over 11 min at a flow rate of 600 μl/min prior to delivery to a SCIEX 6500+ QTrap mass spectrometer (AB Sciex, Framingham, MA, USA). The following MRM transitions were monitored for quantitative purposes: 466 to 105, BZC-dopamine; 488 to 111, ¹³C₆-BZC-dopamine-d₄; 304 to 150, BZC-HVA; 310 to 111, ¹³C₆-BZC-HVA; 394 to 105, BZC-DOPAC; 406 to 111, ¹³C₆-BZC-DOPAC. Automated peak integration was performed using SCIEX Multiquant software version 3.0.2. All peaks were visually inspected to ensure proper integration. Levels of dopamine, HVA, and DOPAC in samples were calculated using calibration curves constructed on the basis of peak area ratio (Panalyte/PI.s.) versus concentrations of internal standard by linear regression. Levels were normalised to protein concentration in the tissue extract.

Protein assay. Protein concentration in tissue homogenates was determined using the Pierce™ BCA Protein Assay Kit (Thermo Fisher Scientific, Waltham, MA USA) as described in the provided kit instructions. Absorbance was measured using a POLARstar Omega plate reader (BMG LABTECH, Offenburg, Germany).

### ELISA for Transgene-Derived Human α-Syn

Dissected striatal tissue from fresh frozen cryosections of all animals was homogenised in a lysis buffer containing protease and phosphatase inhibitors (Roche: 11836153001). Samples were agitated at 4° C. for 30 minutes followed by centrifugation (135000 rpm for 10 minutes at 4° C.) to produce supernatant. Using a 1:500 dilution for a concentration of 0.001 mg/ml, a portion of supernatant was used to determine total protein levels (BCA assay, Pierce, Rockford, IL). Another portion of supernatant underwent ELISA procedures according to the manufacturer's instructions (BioLegend: 844101). The samples were analysed using CLARIOstar systems quantifying the luminescent counts relative to the amount of aSyn. Levels of αSyn were expressed as pg/mg total protein (Pierce™ BCA Protein Assay Kit, Thermo Fisher Scientific, Waltham, MA USA).

### Dopamine Transporter (DAT) Binding

The levels of striatal DAT were be assessed by [125I]-RTI-121 binding autoradiography in cryostat cut sections prepared from 20 μm fresh-frozen tissue. Briefly, thawed slides were placed in binding buffer (2×15 min, room temperature) containing 50 mM Tris, 120 mM NaCl and 5 mM KCl. Sections were then placed in the same buffer containing 50 PM [125I]-RTI-121 (Perkin-Elmer, specific activity 2200 Ci/μmol) for 120 min at 25° C. to determine total binding. Non-specific binding was defined as that observed in the presence of 100 μM GBR 12909 (Tocris Bioscience). All slides were then washed (4×15 min) in ice-cold binding buffer, rinsed in ice-cold distilled water and air-dried. Together with [125I]-microscale standards (Amersham), slides were then apposed to autoradiographic film (Kodak) and left for ˜7 days at room temperature before developing. Autoradiograms were then analysed using MCID software (Image Research Inc, Ontario, Canada). Densitometric analysis of 3 striata from each animal was carried out whereby a reference curve of c.p.m. versus optical density was calculated from β-emitting [14C] micro-scale standards and used to quantify the intensity of signal as nCi/g. Background intensity was subtracted from each reading. Data were then expressed as mean±s.e.m. signal intensity for each treatment group. Non-specific binding was calculated in the same way and subtracted from the total to give specific binding. Non-specific binding was typically found to account for <1% of total binding.

### Immunofluorescence and Stereology

Immunofluorescence: Brains were sectioned frozen in the coronal plane at a thickness of 40 μm on a freezing sliding microtome (Leica Microsystems Inc., Richmond Hill, ON) and 6 series of sections were stored in cryoprotectant (30% glycerol, 30% ethoxyethanol, 40% PBS). Using a single series of midbrain sections, double label immunofluorescence was performed to reveal hemagglutinin (HA)-tagged human aSyn and tyrosine hydroxylase (TH). Briefly, on free floating sections, levels and distribution of TH (sheep anti-TH, 1:1000, Pel Freez, P60101; secondary antibody, Alexa fluor donkey anti-sheep, Fisher Scientific, A21099, 1:500) and HA (rabbit anti-HA, 1:1000; Abcam, AB9110; Alexa Fluor donkey anti-rabbit, 1:500, Fisher Scientific, A21206, 1:500) was evaluated by double label immunofluorescence.

Stereology: Estimates of TH^{+ve} neuronal number with and without human α-syn co-localization within the substantia nigra pars compacta (SNc) was performed using Stereo Investigator software (MBF Bioscience, Williston, VT) according to stereologic principles. Seven or eight sections, each separated by 240 μm from the anterior to the posterior SN, was used for counting each case. Stereology was performed using a Zeiss microscope (AxioImager M2 with Apotome, Carl Zeiss, Canada) coupled to a monochrome digital camera for visualization of tissue sections. The total number of TH+ve neurons with and without human α-syn inclusions was estimated from coded slides using the optical fractionator method. For each tissue section analyzed, section thickness was assessed empirically and guard zones of ˜2 μm thickness were used at the top and bottom of each section. The SNc was outlined under low magnification (5×) and TH+ve neurons counted under 40× magnification. Stereological parameters were empirically determined (i.e. grid size, counting frame size and dissector height) using Stereo Investigator software (MicroBrightfield, VT, USA). The acceptable coefficient of error (CE) was calculated according to the procedure of West and colleagues, known as the Gunderson CE (m=1). Gunderson values <0.10 were accepted.

The results of the counting stereology produced absolute numbers of TH^{+ve} neurons in the SNc to assess neuroprotection. The number of those TH+ve neurons remaining that contain reactivity for human alpha-synculein were also produced to provide an indication of the number of human α-syn expressing TH^{+ve} neurons. A ratio of TH^{+ve}/synuclein^{+ve}: TH^{+ve}/synuclein^{-ve} was then calculated.

### In Vivo Studies

### Study 1. Route of Administration Study in Wild-Type (WT) Rats

In this study the distribution of transgene expression (GFP) was evaluated, 14 days following administration of AAV5-GFP to either the substantia nigra (SN), striatum or cisterna magna. A total of 2 treatment groups with N=5 animals per group (total N=10, female Sprague-Dawley rats, Envigo, USA) were used. On Day 1, animals received, bilateral, 4 ul stereotaxic injections of AAV5-GFP into the SN, 3×3 ul (bilateral) stereotaxic injections into the striatum of AAV5-GFP or 25 ul of AAV5-GFP into the cisterna magna. Groups are indicated in Table 8.

TABLE 8
Groups of in vivo study 1.

| | | Volume / | | Terminal | |
|---|---|---|---|---|---|
| | AAV5-GFP | number of | target | procedures - | |
| Group | (D1) | injection sites | (bilateral) | brain | N |
| 1 | AAV5-GFP, 5 × 10¹⁰ gc/brain | 4 μl × 1 | SN | D14 | 5 |
| 2 | AAV5-GFP, 2.5 × 10¹¹ gc/brain | 4 μl × 1 | SN | D14 | 5 |
| 3 | AAV5-GFP, 2.5 × 10¹¹ gc/brain | 3 μl × 3 | Striatum | D14 | 5 |
| 4 | *AAV5-GFP, 4.5 × 10¹² gc/brain | 25 μl × 1 | ICM | D14 | 5 |

*25 μl of the stock AAV5 (1.8 × 10¹⁴ gc/ml)

For the intra-SN injection, the stereotaxic coordinates were −5.2 mm AP, and −/+2 mm ML relative to Bregma with the needle lowered-7.5 mm below the skull and with the toothbar set at −3.3. Striatal stereotaxic injection coordinates were site 1: +1.3 mm AP, −/+2.8 ML, −4.5 DV; site 2: +0.2 mm AP, −/+3.0 ML, −5.0 DV; site 3: site 2: −0.6 mm AP, −/+4.0 ML, −5.5 DV; with the toothbar set at −3.3. Viral vectors were administered at a rate of 0.5 μl/min and a 5 min wait time allowed after each injection. ICM administrations were made according to a method adapted from Chen et al. 2013 Acta Neurobiol Exp (Wars) 73 (2): 304-11.

On Day 14, rats received an overdose of isoflurane and sacrificed via transcardial perfusion with ice-cold 0.9% saline. Brains were then removed, and the right hemisphere was post-fixed in 4% paraformaldehyde (overnight) and cryoprotected in sucrose solutions. Right hemisphere forebrain and midbrains were then cut on a freezing sliding microtome for histological procedures. The left hemispheres were fresh dissected into regions of interest and individually frozen for molecular analyses.

### Study 2. Mechanism of Action Study in α-Syn KI Rats

In this study, the mechanism of action of two AAV-miSNCA candidates was evaluated in human alpha synuclein KI rats. A total of 3 treatment groups with N=3 animals per group (total N=9, Envigo, USA) were used. On Day 1, all animals received a 3×3 ul unilateral injection of AAV5 into the striatum. The contralateral side served as the control and was injected with formulation buffer in the same manner as the AAV5 injections. Groups are indicated in Table 9.

TABLE 9
Groups of in vivo study 2.

| | Ipsilateral | Contralateral | | Terminal procedures - | |
|---|---|---|---|---|---|
| Group | (AAV5) | (Buffer) | Rat strain | brain samples | N |
| 1 | AAV5-miSCR | formulation buffer | Rat-KI | D43 | 3 |
| 2 | AAV5-miSNCA5 | formulation buffer | Rat-KI | D43 | 3 |
| 3 | AAV5-miSNCA15 | formulation buffer | Rat-KI | D43 | 3 |
| 4 | AAV5- | formulation buffer | Rat-KI | D43 | 3 |
| | miSNCA5 + AAV5- | | | | |
| | miSNCA15 | | | | |

AAV5 titers=total of 2.5×10Exp11 gc/brain, 3 sites intra-striatal
Rat-KI=Envigo human alpha synuclein knock-in rat

Striatal stereotaxic injection coordinates were site 1: +1.3 mm AP, −/+2.8 ML, −4.5 DV; site 2: +0.2 mm AP, −/+3.0 ML, −5.0 DV; site 3: site 2: −0.6 mm AP, −/+4.0 ML, −5.5 DV; with the toothbar set at −3.3. Viral vectors were administered at a rate of 0.5 μl/min and a 5 min wait time allowed after each injection.

On Day 43, all rats received an overdose of isoflurane and were transcardially perfused with ice-cold 0.9% saline. Brains were then removed as rapidly as possible and left and right hemispheres were divided. In all animals from each group, the following regions were freshly dissected, separately for left and right hemispheres, frozen on dry ice and stored at −80° C. for molecular analyses: prefrontal cortex, striatum, hippocampus, hypothalamus, thalamus, posterior cortex, cerebellum, ventral midbrain and brainstem.

### Study 3. Study in AAV-Syn Rat Model

This study was designed to assess the ability of two artificial miRNAs targeting SNCA mRNA (encoding aSyn) to protect dopaminergic function in the AAV1/2-hA53T-aSyn rat model of Parkinson's disease. The model involves injection of the WT rat unilaterally with an AAV1/2 human A53T α-syn (AAV1/2-hA53T-aSyn) and an AAV5-miRNA (either miSNCA or an unrelated (control) miRNA). There were two groups in terms of the injections of these two viruses: a co-injection and a sequential injection group. In the co-injection groups the two viruses were injected on day 1 (groups 1-4 in Table 10) and in the sequential groups AAV1/2-hA53T-aSyn was injected on day 1, whereas the AAV5-miSNCAs were injected on day 14 (groups 5-8 in Table 10). On the indicated days in Table 10 a single virus or a combination was administered unilaterally into the right substantia nigra according to stereotaxic techniques. Behavioral assessment was conducted in the cylinder test, to assess forelimb asymmetry, prior to surgery (baseline, D-3) and on D14, D21, D42 and D56 (2, 3, 6 and 8 weeks following AAV administration). Groups are indicated in Table 10.

TABLE 10
Groups of in vivo study 3.

| | | | Behaviour | | |
|---|---|---|---|---|---|
| | Viral vector -1 | | D −3, 14, | Terminal | |
| | | | 21, 42, 56 | procedures, | |
| | | Viral vector -2 | cylinder | D57 | |
| Group | intra-SN, 2 ul volume | | test | kill, brain | N |
| 1 | D1: AAV1/2-EV | D1: AAV5-unrelated miR | ✓ | ✓ | 12 |
| | | (6E10 gc/brain) | | | |
| 2 | D1: AAV1/2-hA53T- | D1: AAV5-unrelated miR | | | 12 |
| | aSyn | (6E10 gc/brain) | | | |
| 3 | D1: AAV1/2-hA53T- | D1 AAV5-miSNCA5 | ✓ | ✓ | 12 |
| | aSyn | (6E10 gc/brain) | | | |
| 4 | D1: AAV1/2-hA53T- | D1: AAV5-miSNCA15 | ✓ | ✓ | 12 |
| | aSyn | (6E10 gc/brain) | | | |
| 5 | D1: AAV1/2-EV | D14: AAV5-unrelated miR | ✓ | ✓ | 12 |
| | | (6E10 gc/brain) | | | |
| 6 | D1: AAV1/2-hA53T- | D14: AAV5-unrelated miR | ✓ | ✓ | 12 |
| | aSyn | (6E10 gc/brain) | | | |
| 7 | D1: AAV1/2-hA53T- | D14: AAV5-miSNCA5 | ✓ | ✓ | 12 |
| | aSyn | (6E10 gc/brain) | | | |
| 8 | D1: AAV1/2-hA53T- | D14: AAV5-miSNCA15 | ✓ | ✓ | 12 |
| | aSyn | (6E10 gc/brain) | | | |

AAV1/2-hA53T-αSyn=AAV1/2 human A53T alpha-synuclein, 3×10¹² gp/ml
AAV1/2-EV=empty αSyn vector, 3×10¹² gp/ml
AAV5-miSNCA-5=6×10⁰¹ gc/brain
AAV5-miSNCA-15=6×10⁰¹ gc/brain
AAV5-unrelated miR-control, 6×10⁰¹ gc/brain
SN=substantia nigra
D=day
Groups 1-4, are co-injection of AAVs on D1
Groups 5-8, are sequential injection of AAVs on D1 and D14

On D57, animals were sacrificed for postmortem assessments. Blood, samples were collected, processed and stored as required.

### Primary Endpoints of the Study Included:

assessment of forelimb asymmetry (via cylinder test).
quantification of striatal dopamine and metabolite levels (via LC-MS/MS).
quantification of dopamine transporter (via autoradiography).
quantification of striatal transgene-derived aSyn levels (via ELISA)

### Optional Endpoints Included:

quantification of tyrosine hydroxylase positive (TH+ve) cells in the substantia nigra with and without co-expression with human aSyn (via double label immunofluorescence)
qualitative assessment of activation status of microglia in the substantia nigra by Iba-1 immunoreactivity (via immunofluorescence)

Sacrifice and sample collection were done as follows: animals were deeply anaesthetized with isoflurane and then killed via exsanguination by way of transcardial perfusion with ice-cold 0.9% saline containing 0.2% heparin. Brains were placed, ventral up, into an ice-cold stainless steel rat brain matrix and were first cut in the coronal plane at the level of the hypothalamus. The rostral portion of the brain, including the entire striatum, was immediately frozen in isopentane chilled to −42° C. and later sectioned for DAT autoradiography and dissected for the quantification of levels of dopamine and metabolites of dopamine (HVA and DOPAC) by LC-MS/MS and levels of human aSyn by ELISA. Tissue were stored in a locked freezer at −80° C. Additional regions of interest (including additional striatal dissections) were collected according to table 11 for molecular assays.

TABLE 11
Brain regions of interest and corresponding assays and sample handling.

| Region of interest | Assay | Buffers used | Format |
|---|---|---|---|
| striatum | autoradiography | None, cryosectioned | on-slide |
| striatum | LCMS | See LCMS method | supernatant |
| | | below | |
| striatum | ELISA | RIPA, protease and | share aliquots |
| | | phosphatase inhibitors | between assays |
| striatum | TR-FRET | RIPA, protease and | |
| | | phosphatase inhibitors | |
| striatum | Q-PCR (DNA) | None, cryosectioned | In tube |
| | | and dissected | |
| striatum | RT-QPCR (miRNA, | None, cryosectioned | In tube |
| | RNA) | and dissected | |
| striatum | backup | None, cryosectioned | In tube |
| | | and dissected | |

The remainder of the caudal portion of the brain, including the mesencephalon, was immersed in 4% paraformaldehyde (PFA) for 48 hours for fixation followed by cryoprotection in graded sucrose solutions (15 to 30% sucrose). Tissue prepared in this manner was used for quantification of dopamine neuron numbers in the SNc via immunohistochemistry of tyrosine hydroxylase and unbiased stereology.

Study 4. Phenotypic Rescue of Motor Phenotype in C. elegans PD Model

In this study, the effect of expression of miSNCA candidates on phenotypic rescue of altered motor behavior in a C. elegans PD model (OW40; van Ham et al 2008 PLOS Genet 4 (3): e1000027) was evaluated. In this model, human α-syn is overexpressed in the body wall muscle of C. elegans. This α-syn overexpression causes the slowing of the movement of the worms when compared to the control worms. The effect of decreasing SNCA mRNA levels, thereby decreasing the α-syn protein levels by RNAi, was studied using the full length SNCA gene or our miSNCA constructs. The double stranded RNA with one of these constructs were introduced to the organism by feeding. The C. elegans OW40 worms were fed by E. coli overexpressing either the empty T444T plasmid, as negative control, or the full length SNCA gene or one of our miSNCA candidates (miSNCA5, miSNCA13 or miSNCA15) at different stages of their life: Larval stage 1 (L1), Larval Stage 4 (L4) and Day 1 of their adulthood. The treatment experiments were repeated at 25° C. and at 15° C. degrees. Following treatment on day 1, day 4 and day 8 of their adulthood, the worms were video tracked using a high throughput tracking set up to measure their movement (speed as μm/s) (Perni et al 2018 Journal of Neuroscience Methods 306 57-67).

Additional read-outs were RT-qPCR of the SNCA mRNA and α-syn protein levels using western blot analysis. The primer sequences used for RT-qPCR of the SNCA mRNA are provided in Table 12.

TABLE 12
Primers used for RT-qPCR of the SNCA mRNA

| Primer | Type of | | |
|---|---|---|---|
| for | gene | Forward primer | Reverse primer |
| SNCA1 | transgene | GACTTTCAAAGGCCAAGGAG | GGAGCCTACATAGAGAACA |
| | | (SEQ ID NO. 70) | C |
| | | | (SEQ ID NO. 71) |
| SNCA2 | transgene | ATTGCAGCAGCCACTGGCTTTG | GGCTTCAGGTTCGTAGTCTT |
| | | (SEQ ID NO. 72) | G |
| | | | (SEQ ID NO. 73) |
| PMP3 | House- | CACTCATCTCTATGACGACGTT | CACCGTCGAGAAGCTGTAG |
| | keeping | TC | A |
| | gene | (SEQ ID NO. 74) | (SEQ ID NO. 75) |

For detection of α-syn protein levels western blot analysis was used. For this purpose, the proteins were extracted using RIPA buffer and Tissue lyzer (Qiagen). Similar protein amounts from different treatment conditions were loaded onto the SDS PAGE and western blot was performed using anti human α-syn antibody (Table 12) for detecting the α-syn levels. Tubulin was used for normalization and detected using anti tubulin antibody (Table 13).

TABLE 13
Antibodies used for C. elegans Western Blot analysis

| Antibody name | Catalog # | Type of protein |
|---|---|---|
| Alpha synuclein | 18-0215 (Invitrogen) | Target protein |
| Tubulin | T6074 (sigma) | Housekeeping |
| | | normalization |

### Results:

### In Vitro Experiments

In Vitro Silencing Efficacy of Artificial miSNCA Constructs

To evaluate the miSNCA knockdown efficacy of the miSNCA constructs in vitro, HEK293T cells were co-transfected with Renilla luciferase reporters encoding the SNCA gene. The Firefly luciferase (FL) gene was expressed from the same reporter vector and served as an internal control to correct for transfection efficiency. In the first screening HEK cells were co-transfected with Jan. 10, 1950 or 250 ng of each of the miSNCA constructs and Dual Luc reporter carrying SNCA gene. Out of 17 miSNCA constructs designed to target SNCA gene, miSNCA2, miSNCA5, miSNCA7, miSNCA12, miSNCA13, miSNCA15 and miSNCA16 induced dose-dependent decrease in the RL/FL ratio. To further determine the potency, above-mentioned constructs were further used for titration experiments. The constructs were co-transfected into HEK293T cells in different concentrations; 0.1, 1, 10, or 100 ng with 10 ng of SNCA luciferase reporter plasmid. According to these results, the transfections at 100 ng of miSNCA plasmids showed at least 50% decrease for all the miSNCA candidates used in the titration experiments (FIG. 3). miSNCA5, miSNCA13 and miSNCA15 were chosen for further testing in different models because of their relatively higher efficacy in decreasing mSNCA levels.

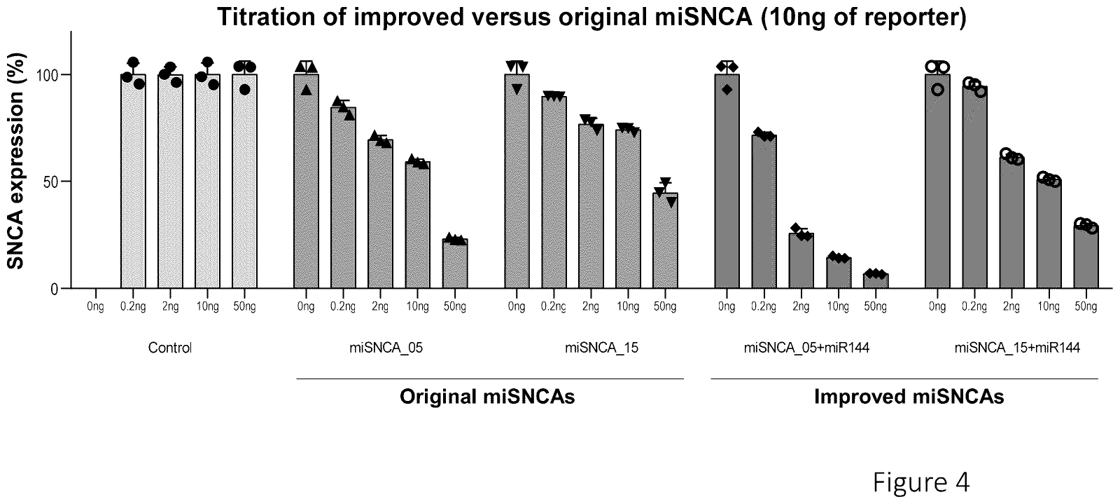

To improve the efficacy of the miSNCA candidates, the native companion of mir451, a modified miR144 was added into the scaffold of the miSNCA5 and miSNCA15 (FIG. 2 B) (SEQ ID NOs. 32-33). These constructs were designed in a way that the modified miR144 is added into the scaffold on the 5′ end of the miR451 carrying miSNCA candidates. To evaluate the efficacy of the original and improved miSNCA candidates Dual Luciferase assays were performed. The constructs (miSNCA5 (SEQ ID NO. 25), miSNCA15 (SEQ ID NO. 29), miSNCA5+miR144 (SEQ ID NO. 31), miSNCA15+miR144 (SEQ ID NO. 32) and control miRNA were co-transfected into HEK293T cells in different amounts per 24-well; 0.1, 1, 10, or 100 ng with 10 ng of SNCA luciferase reporter plasmid. According to these results the efficacy of miSNCA5 and miSNCA15 was improved at least 2-3 times (FIG. 4).

### Lowering of Endogenous SNCA Expression in Transfected Cells

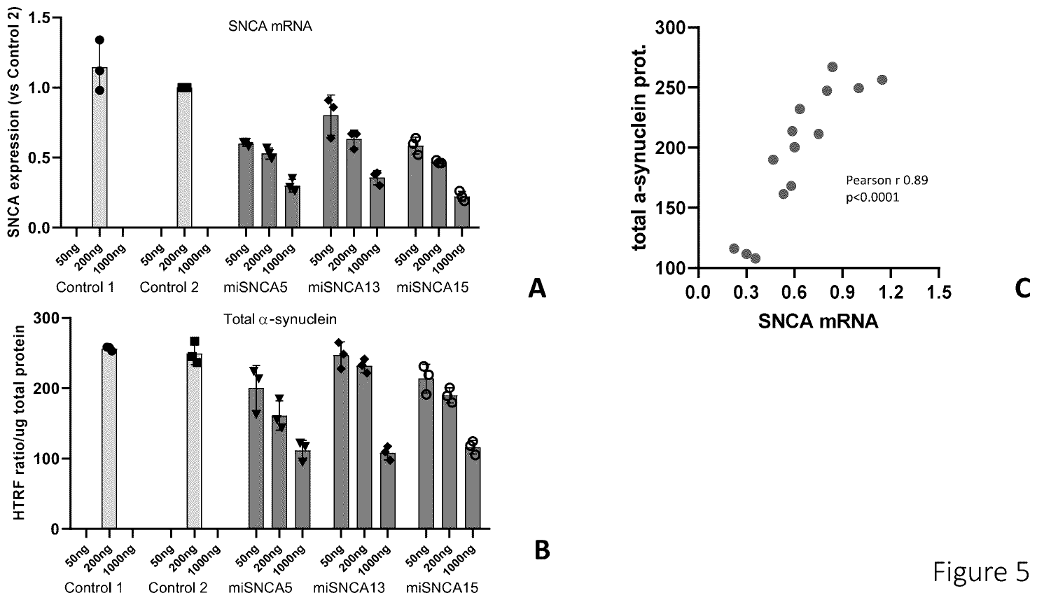

miSNCA5, miSNCA13 and miSNCA15 constructs were chosen for testing the knockdown of SNCA mRNA expression in cells. The knockdown of the endogenous SNCA gene expression in HEK293T cells was confirmed by RT-QPCR on transfected cells. Transfection of 50-200-1000 ng of miRNA plasmid resulted in a decrease of SNCA mRNA expression of <40% by all miSNCA candidates tested. The results were also consistent at the protein levels a dose-dependent decrease in α-syn levels measured by HTRF. (FIG. 5)

Expression Levels of miRNAs in Transduced Cells (Small RNA Sequencing Data)

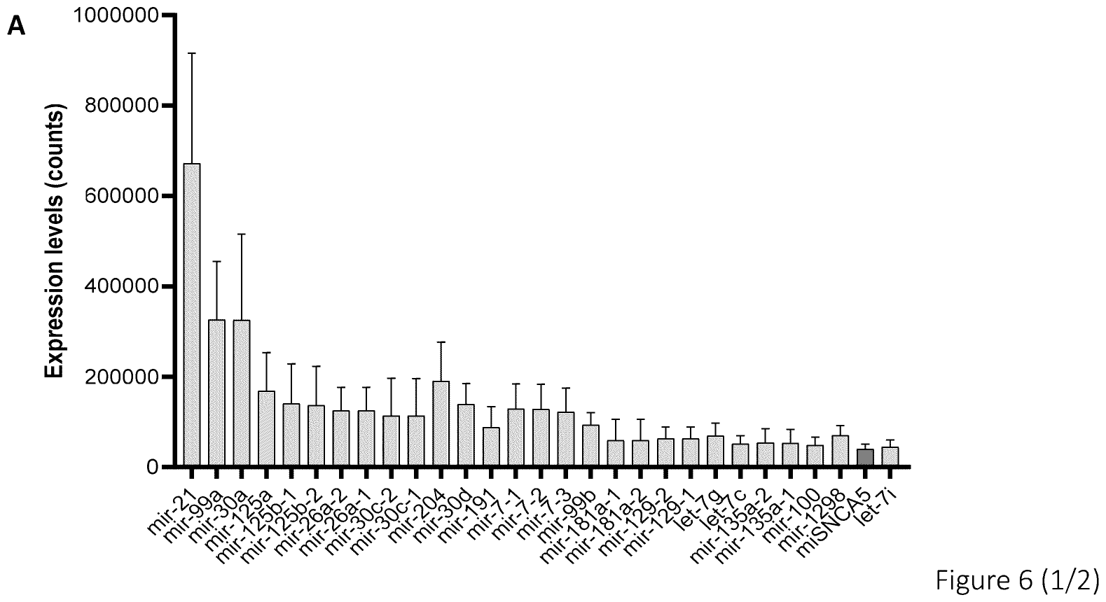

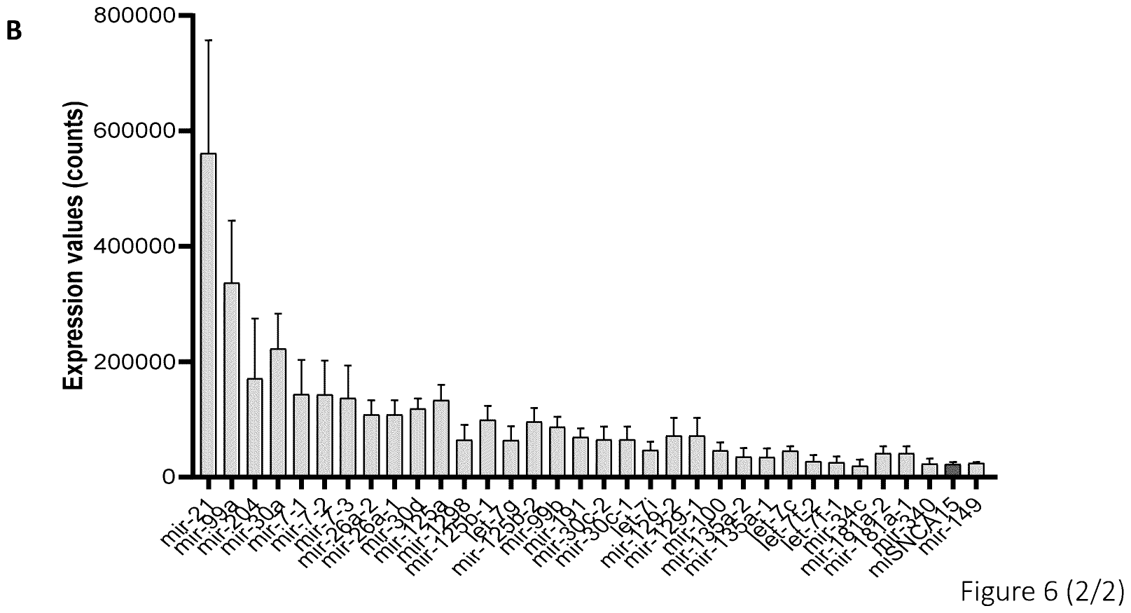

The expression level of the mature miRNAs was quantified based on the number of the total reads annotated by using miRBase and the pre-miRNA sequence of interest. Expression levels of the top 30 and 35 most expressed miRNAs in DA neurons transduced with HEK produced AAV5-miSNCA at MOI of 10⁶/cells was obtained (FIG. 6), miSNCA5 (FIG. 6A) and miSNCA15 (FIG. 6B) expression levels were well within endogenous miRNA levels.

Processing of miSNCA Constructs Upon Transfection in Cells (NGS Data)

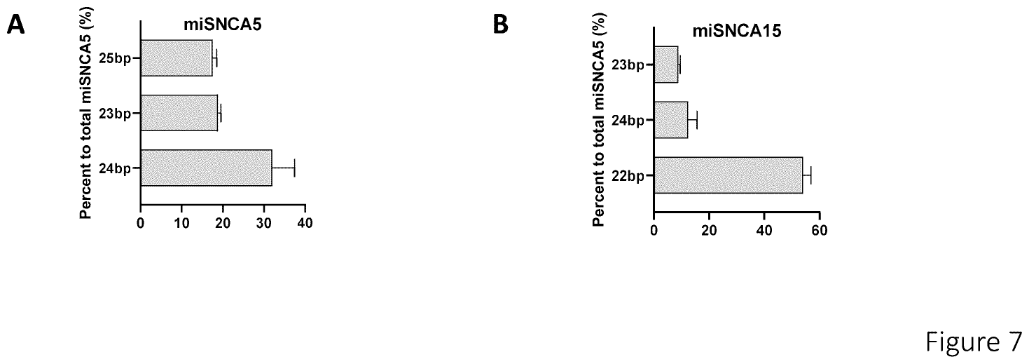

The processing of miRNAs was also investigated by alignment of the reads to the pre-miRNA sequences. The length of the most abundant form for miSNCA5 was 24 nts, followed by 23 and 25 nts (FIG. 7A); for miSNCA15, it was 22nts, followed by 24nts and 23 nts (FIG. 7B).

### AAV5-miSNCA Transduction in Human Cells

To investigate the ability of AAV5-miSNCA5 and AAV5-miSNCA15 to transduce and deliver the packaged expression cassette, DA neurons or forebrain neurons and/or LUHMES-derived DA neurons are transduced at various Multiplicity of Infections (MOI); 10⁴, 10⁵, 10⁶ and 10⁷. The vector DNA levels are measured and there should be a dose-dependent vDNA level increase in these cells. The RNA is isolated from the transduced cells and mRNA levels of SNCA is measured using RT-qPCR Syber Green assays. A dose dependent mRNA decrease of SNCA levels in these transduced cells is expected.

Processing of miSNCA Constructs from Baculovirus Produced Constructs (NGS Data)

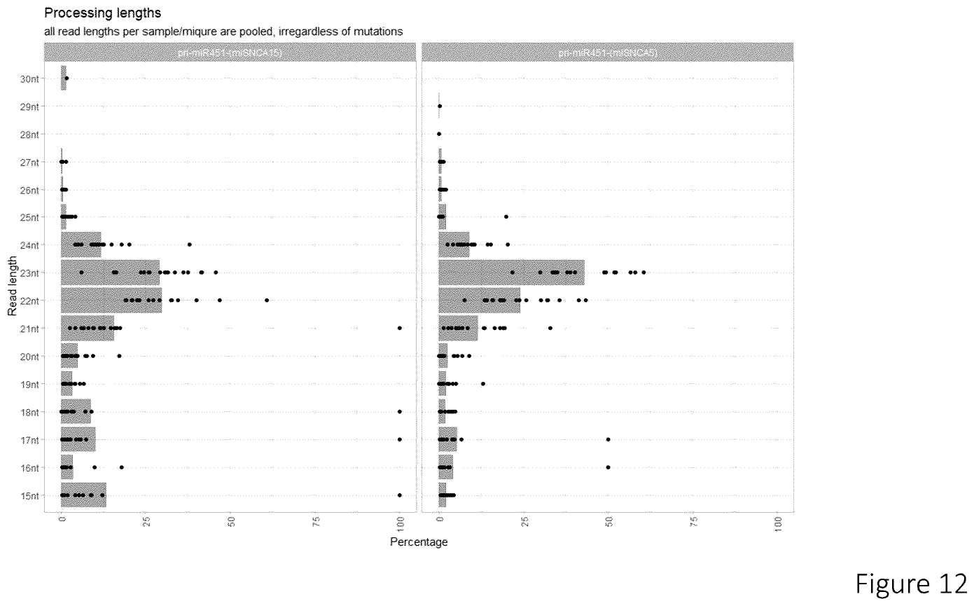

The processing of miRNAs extracted from rat brain tissue from in vivo study #2, group #4 was also investigated to evaluate the processing of the baculovirus produced AAV5-miSNCAs. Small RNA sequencing was done on these samples and the data was aligned with the reads to the pre-miRNA sequences. The length of the most abundant form for miSNCA5 was 23 nts, followed by 24 and 25 nts; and for miSNCA15 it was 22nts, followed by 24 nts and 23 nts (FIG. 12).

### In Vivo Studies

### Study 1. Different Routes of Administration of AAV5-GFP in WT Rats Showed Good Coverage in Target Regions Affected in Parkinson's Disease.

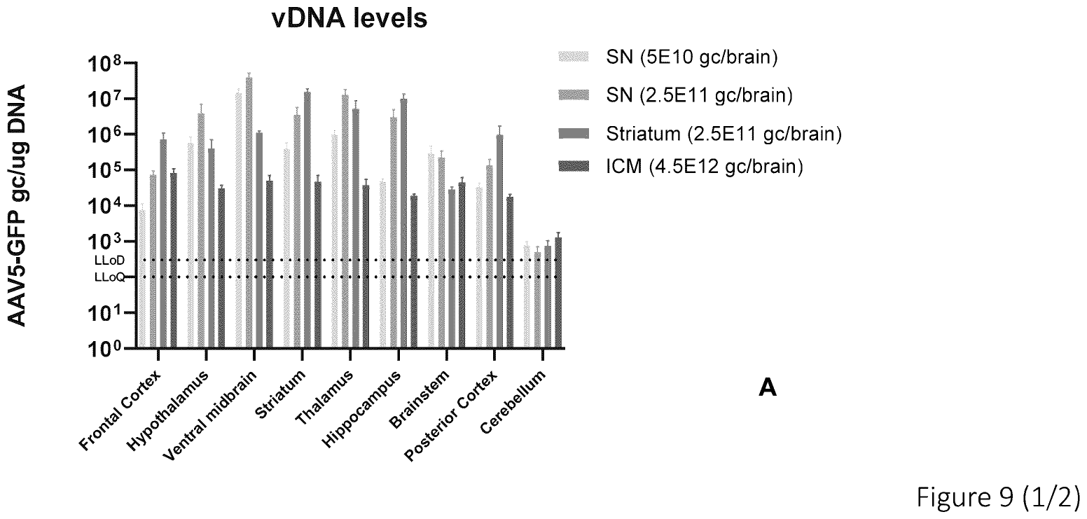

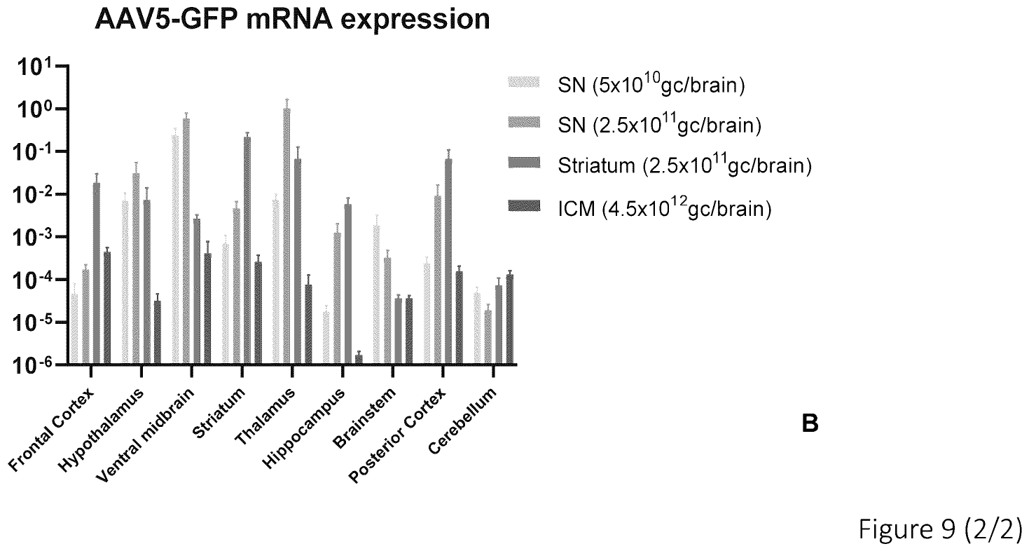

To address the adequacy of the AAV5 vector to deliver miSNCA candidates to target brain regions, the coverage of brain areas that show significant α-syn pathology in Parkinson's disease (brainstem, midbrain, cortex) was assessed after AAV5 administration. Different routes of administration were tested: substantia nigra (SN), striatum or cisterna magna. GFP was used as a reporter gene. AAV5-GFP injected in SN, at the two doses examined, or in striatum, at the single dose examined, showed adequate biodistribution in target areas, as evaluated by AAV-GFP vDNA levels in the brain (FIG. 9.A) and corresponding GFP mRNA expression (FIG. 9.B). AAV5-GFP injected in cisterna magna (directly into the cerebrospinal fluid) lead to equal coverage of all the brain regions examined, although to a lesser extent. It was thus concluded that AAV5 is an adequate vector to deliver miSNCA candidates to brain regions of interest for Parkinson's disease treatment.

Study 2. AAV5-miSNCA Candidates Lowered Human SNCA mRNA Expression in α-Syn KI Rats

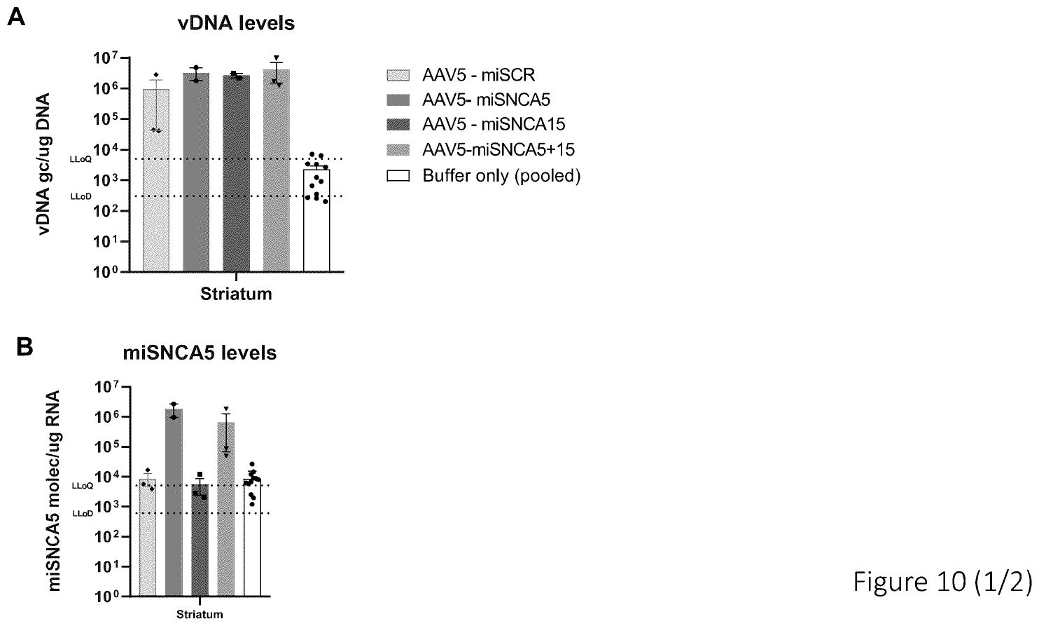

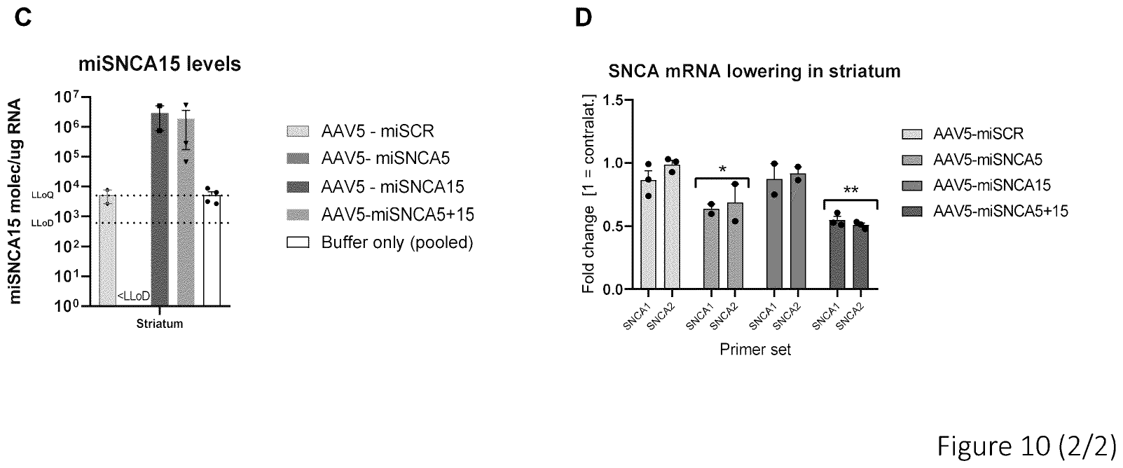

To evaluate the capacity of two of the designed candidates (miSNCA5 and miSNCA15) in lowering human SNCA mRNA expression, AAV5-miSCR (non-targeting scramble control), AAV5-miSNCA5 or AAV5-miSNCA15 were injected into the left striatum of adult α-syn KI rats. One group was injected with an equivalent dose of combined AAV5-miSNCA5 and AAV5-miSNCA15. The miSNCA13 was excluded from in vivo studies because it targets a region outside of the humanized part of SNCA KI rat model, and it has 3 mismatches to the WT rat SNCA gene. The right striatum received formulation buffer injection, which served as additional control. At the single dose used, vDNA was detected in the AAV5 injected brain hemispheres, while in the control hemispheres, vDNA levels were below the lower limit of quantification (LLOQ) (FIG. 10.A).

Transduction led to expression of miSNCA candidates 5 and 15 or a combination of both, in a vector-specific manner (FIGS. 10.B and C): miSNCA5 was detected only in the AAV5-miSNCA5 injected hemisphere, miSNCA15 only in the AAV5-miSNCA15 injected one, and miSNCA5 and miSCNA15 in the AAV5-miSNCA5+AAV5miSNCA15 injected group. At the single dose used, AAV5-miSNCA5 and AAV5-miSNCA5+AAV5-miSNCA15 were effective in reducing SNCA mRNA expression in the injected striatum, as evaluated by two different RT-QPCR SNCA assays (primer set SNCA1 and primer set SNCA2), compared to the control striatum (FIG. 10.D). This study supports the mechanism of action of AAV5-miSNCA candidates to reduce human SNCA mRNA expression and α-syn toxicity for the treatment of Parkinson's disease.

### Study 3. Study in AAV-Syn Rat Model

Different AAV5-miSNCA candidates were tested in the AAV-Syn rat model.

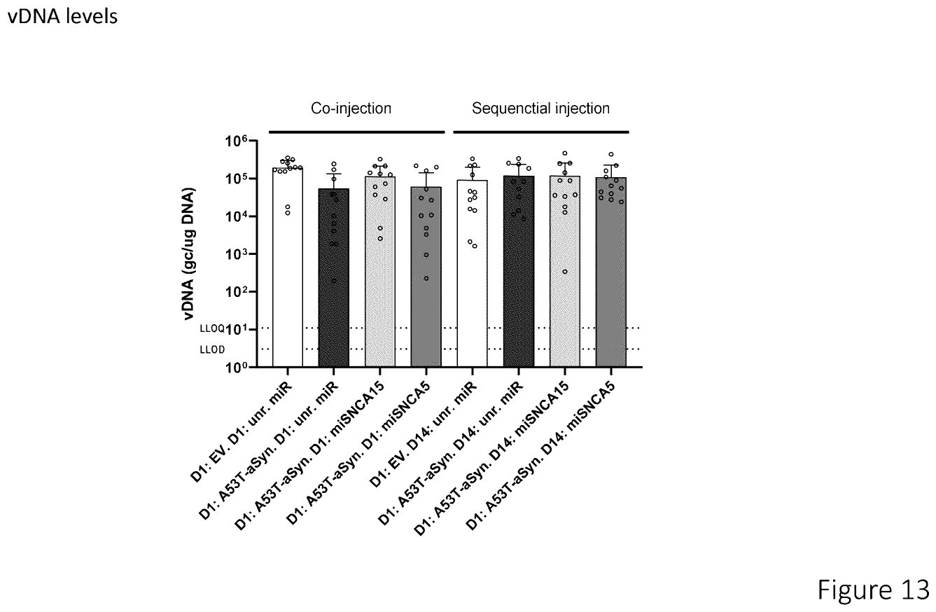

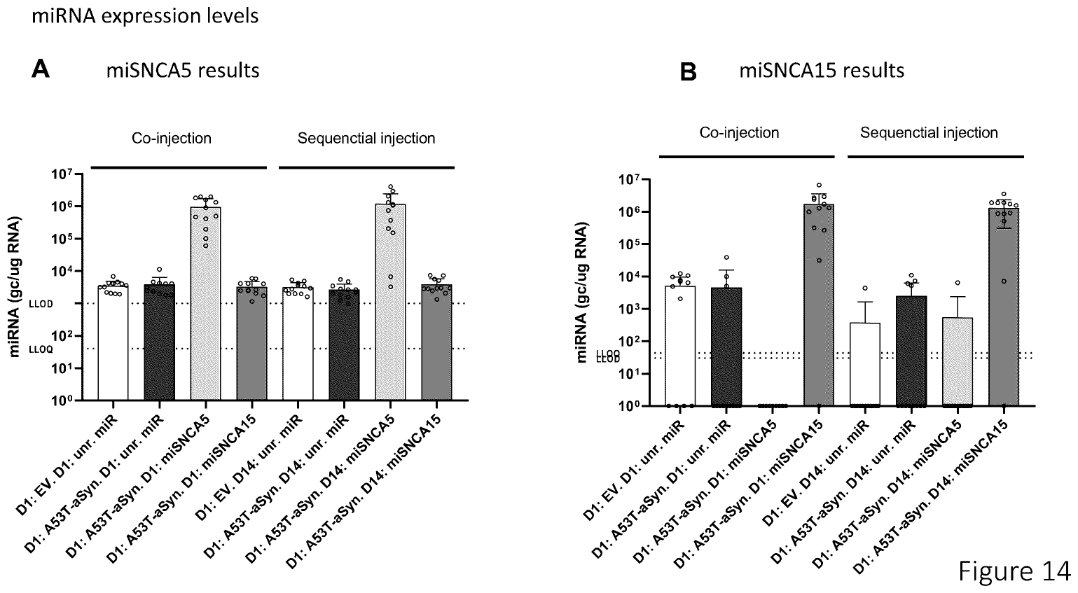

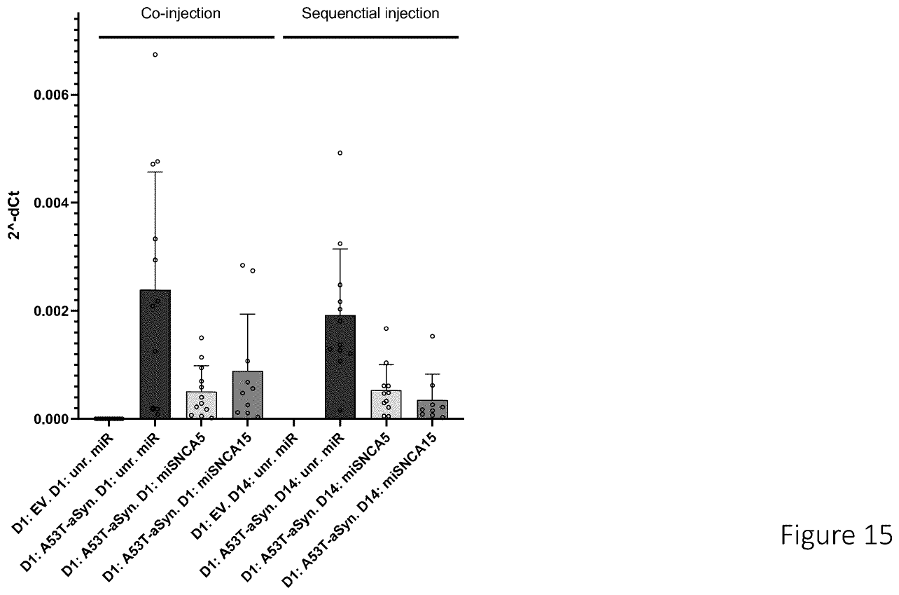

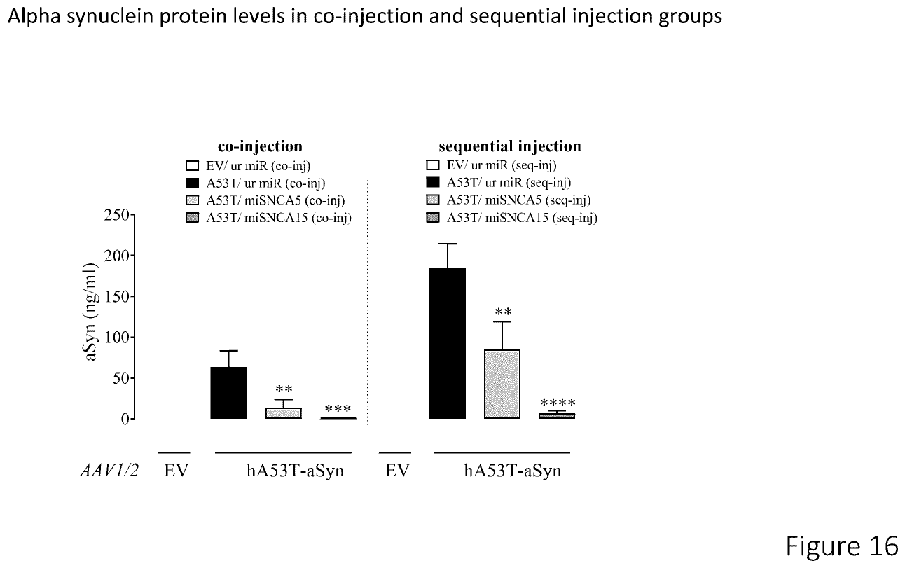

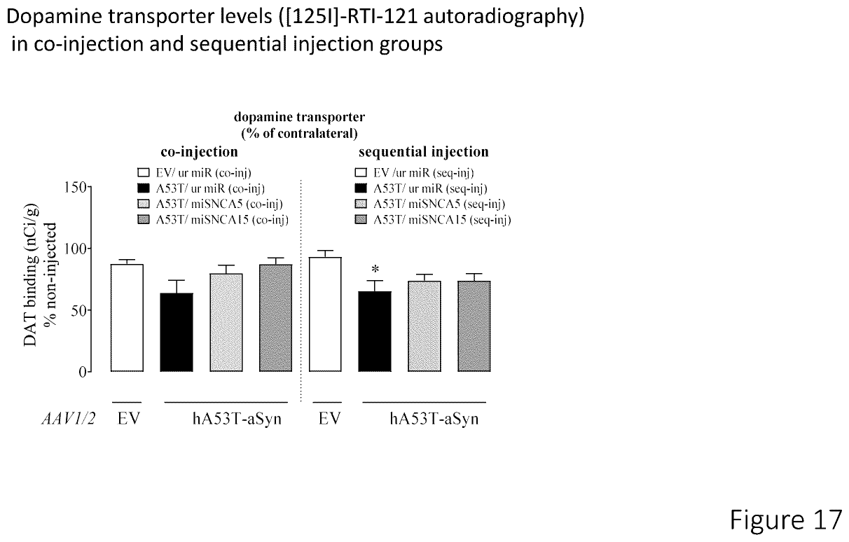

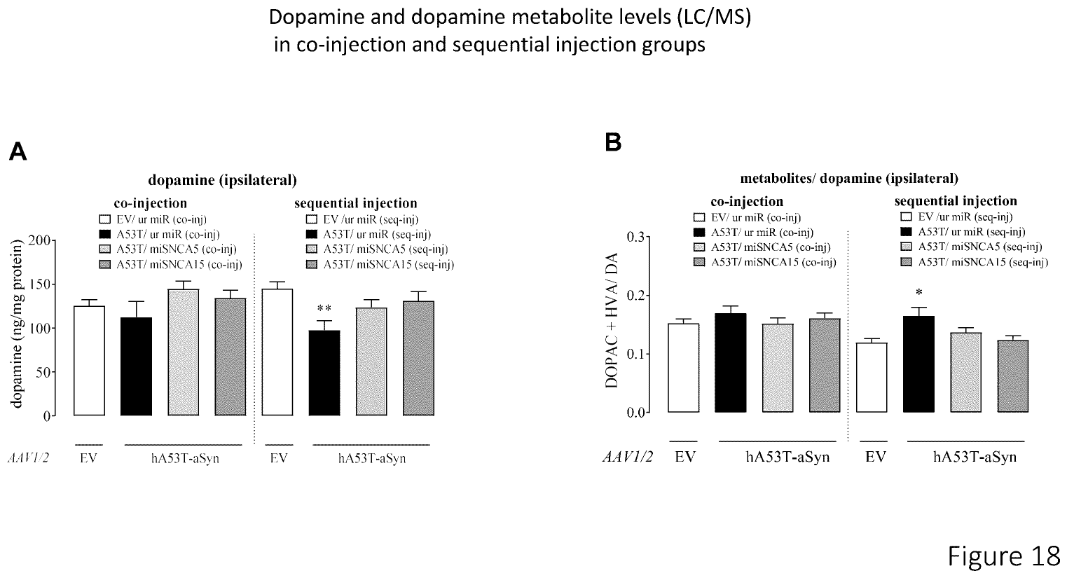

To show the in vivo proof of concept of lowering SNCA levels, thereby improving the motor phenotype human A53T variant of α-syn viral overexpression, rat PD model was used (AAV1/2-hA53T-aSyn). The right substantia nigra (SN) received unilateral injections for both the AAV1/2-hSNCA and AAV5-miSNCA virus. In both the co-injection and the sequential injection groups, at the single dose used, vDNA was detected in the AAV5 injected brain hemispheres in striatum (FIG. 13), while in the control hemispheres (left striatum), vDNA levels were below the lower limit of quantification (LLOQ) (not shown). Transduction led to expression of miSNCA5 and miSNCA15, in a vector-specific manner (FIGS. 14A and B): miSNCA5 was detected only in the AAV5-miSNCA5 injected hemisphere, miSNCA15 only in the AAV5-miSNCA15 injected one, and neither was detected in the other samples from the negative control groups. In A53T-aSyn animals, at the single dose used, AAV5-miSNCA5 and AAV5-miSNCA15 were effective in reducing SNCA mRNA expression in the injected site striatum, as evaluated by Taqman RT-qPCR assays (SNCA2 primer and probe combination), compared to the control striatum injected with unrelated miRNA (solid black bars) (FIG. 15). The miSNCA expression also showed lowering at the protein level reflected by reduced α-syn protein levels measured by ELISA (FIG. 16). Dopamine transporter deficits measured by [125I]-RTI-121 autoradiography was apparent in the A53T-aSyn animals sequentially injected with a control miRNA (unrelated miR, group 6 in Table 10), as in PD patients, and were corrected in the miSNCA treated groups (groups 7 and 8 in Table 10) (FIG. 17). Related to the metabolite changes in this model, correction of hA53T-aSyn-induced striatal dopamine deficits by miSNCA candidates were observed. FIG. 18A shows the dopamine levels in study groups and FIG. 18 B shows the (DOPAC+HVA)/DA levels measured by LC/MS.

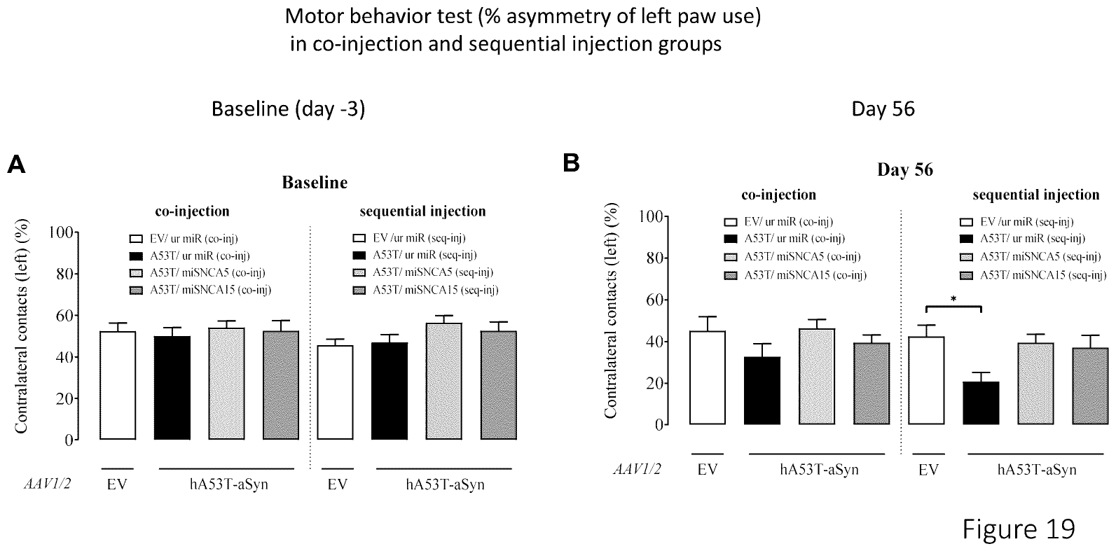

The motor behavior measured by percent asymmetry of left paw use was significantly improved in the sequentially injected groups which received miSNCA5 or miSNCA15 treatment on day 56 compared to the baseline levels (FIGS. 19A and B).

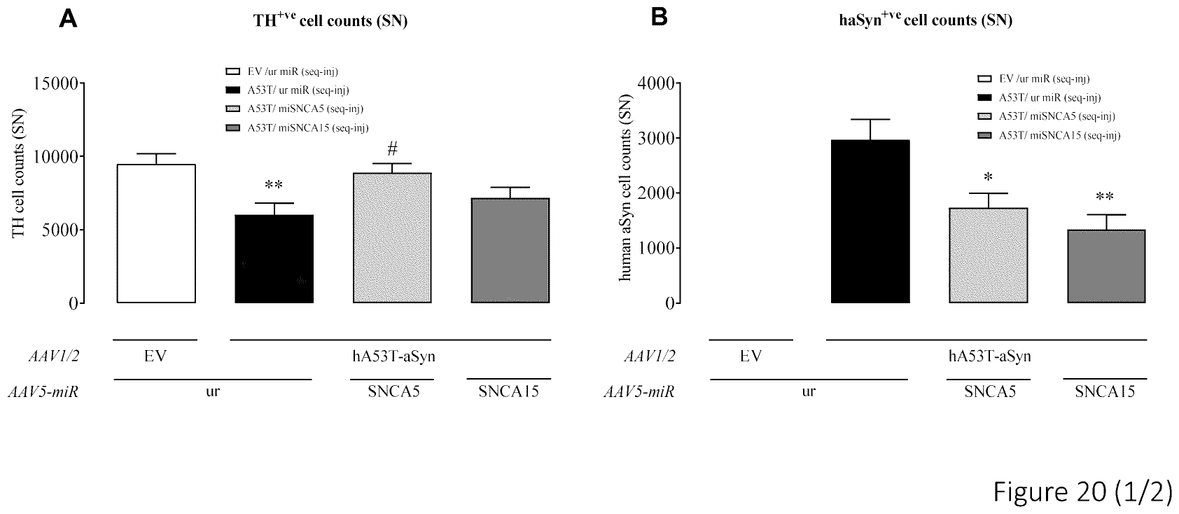

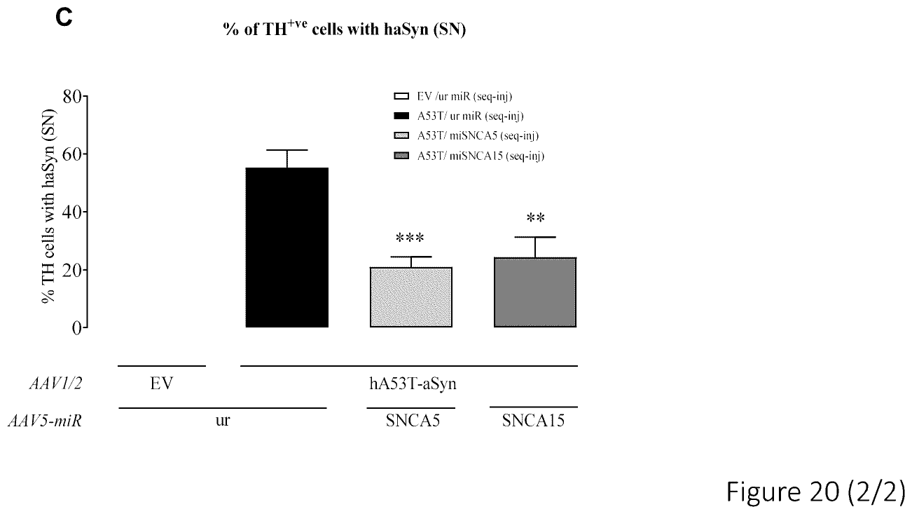

The molecular, biochemical and motor behavioral results were supported with histological observations. Immunostaining and quantification of dopaminergic (TH positive) and α-syn positive neurons in the substantia nigra showed that both AAV5-miSNCA candidates (sequential injection groups) rescued dopaminergic (TH) neuron cell loss (FIG. 20A) and reduced human a-syn cell counts (FIG. 20B). This was reflected by a reduction of the percentage of positive dopaminergic (TH) cells that expressed α-syn (FIG. 20C). A decrease in the inflammation in this model, assessed by Ibal immunoreactivity in the substantia nigra, is also expected.

Overall, AAV5-miSNCA recovered the disease phenotype in the AAV-Syn rat model, improved motor phenotype and rescued the molecular and neurochemical alterations, proving that miSNCA treatment is an effective therapy to reduce α-syn toxicity.

Study 4. Phenotypic Rescue in C. Elegans PD Model with miSNCA Candidate Sequences

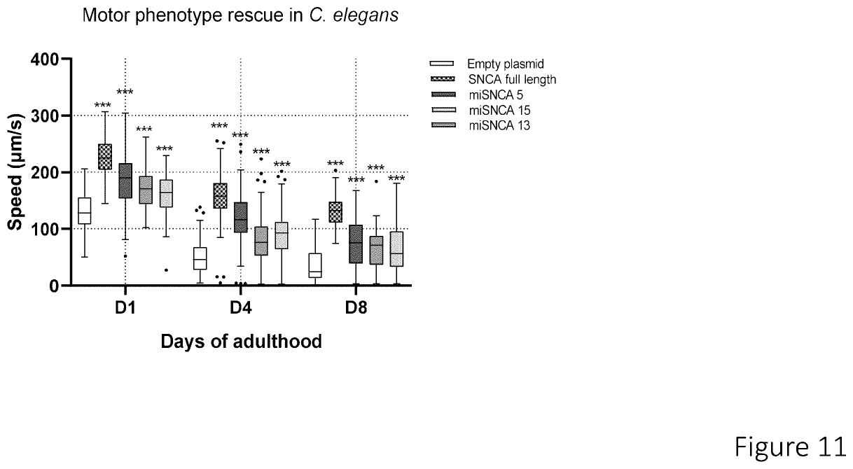

In order to compare the speed of movement between the C. elegans worms fed by different plasmid-expressing E. coli, 100 worms per condition were video tracked. According to the results the worms that were fed by full length SNCA-expressing or miSNCA-expressing E. coli showed an improvement in their speed of movement compared to worms that were fed by the empty plasmid transformed E. coli (FIG. 11). Worms treated with full length SNCA or miSNCAs had an increased speed compared to the untreated worms on all days that their movements were tracked. These results show that decreasing the expression of SNCA gene, thereby reducing the α-syn levels, improves the motor phenotype in this C. elegans PD model. Moreover, the results prove that treatment of worms at different life stages is possible.

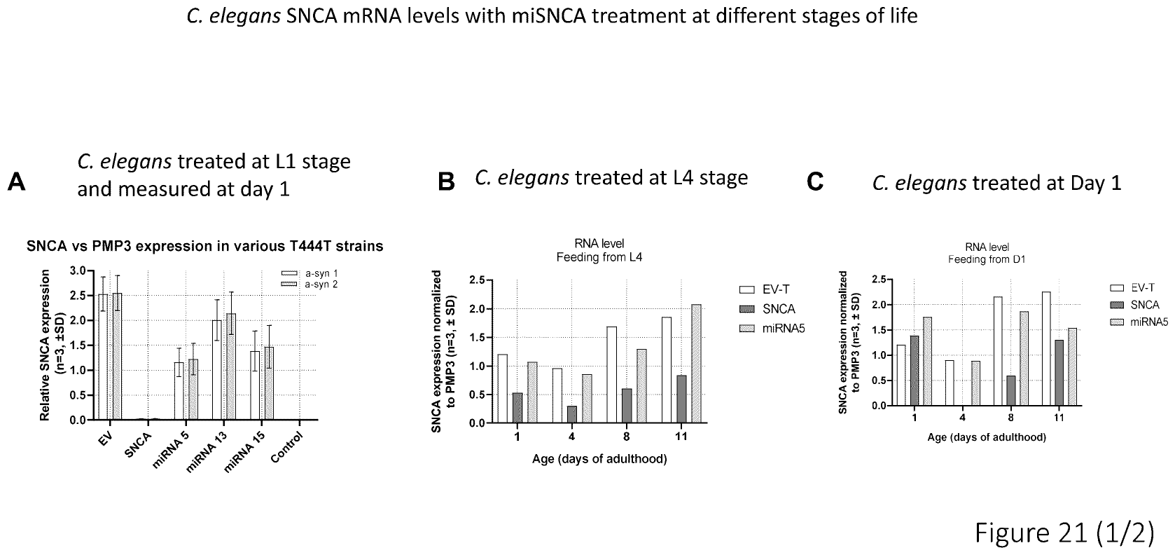

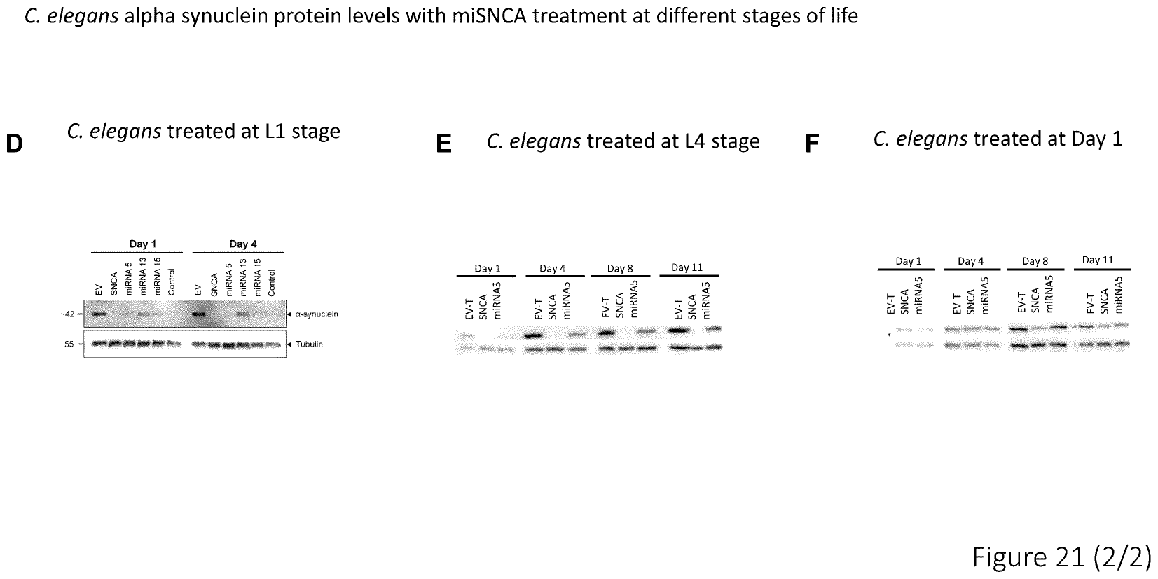

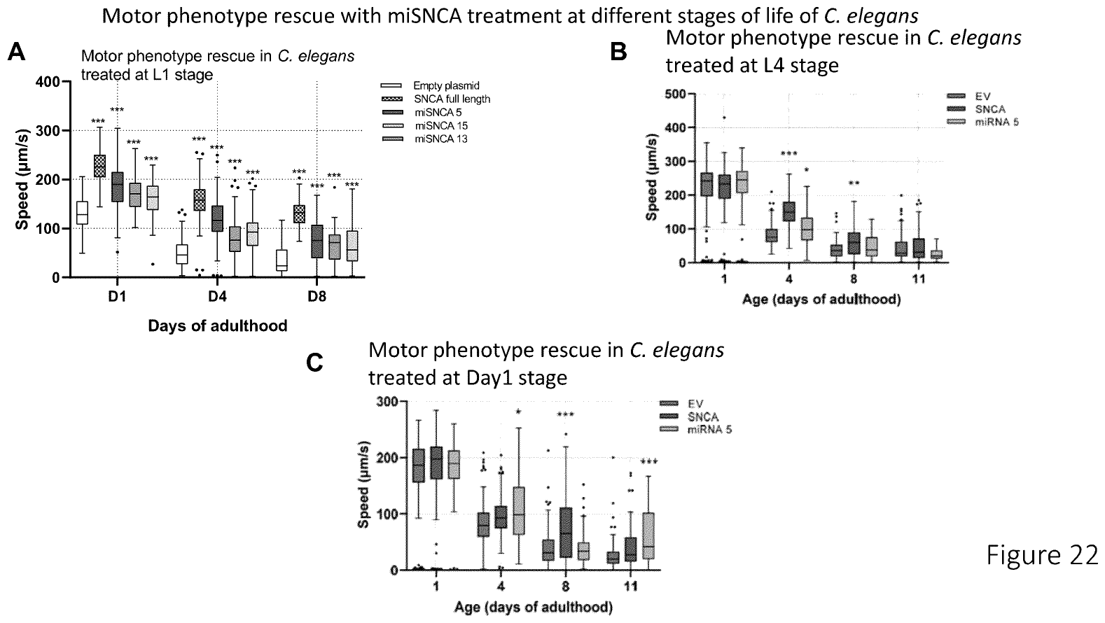

Treatment with miSCNA decreased the SNCA mRNA levels (FIGS. 21A-C) and α-synuclein protein levels (FIGS. 21 D-F) in C. elegans PD model when they are treated at larval or adult stages. In line with this, miSNCA treatment rescued the motor phenotype behavior in this model as shown in FIG. 22 as improvement of speed of swimming in the miSNCA treated worms as compared to the negative control worms (EV-treated worms).

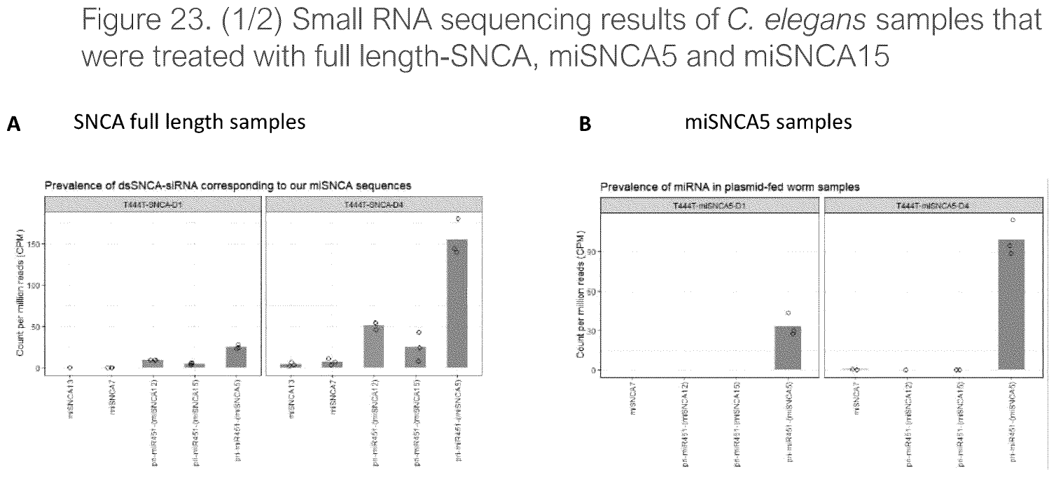

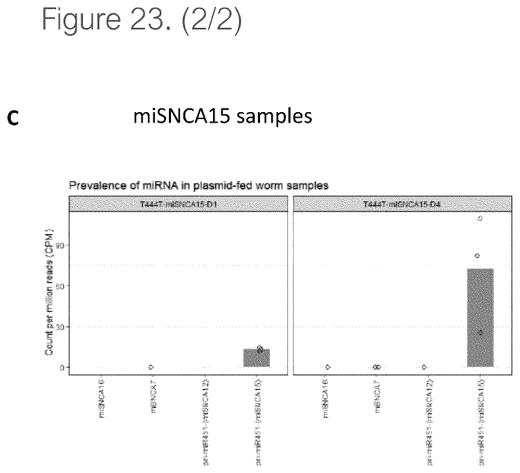

Small RNA sequencing performed in C. elegans samples collected from worms that were treated 5 with miSNCA5, miSNCA15 and full length SNCA, proves the presence of the correctly processed miSNCA candidates in the samples (FIG. 23). The miSNCA5 and miSNCA15 sequences, as well as other miSNCA sequences (e.g. miSNCA7, miSNCA12 and miSNCA13) were detected from the SNCA full length treated samples.

1. A nucleic acid comprising a nucleic acid sequence encoding an RNA, wherein an RNA sequence comprised in said RNA is substantially complementary to a target sequence of an alpha-synuclein (α-syn) gene (SNCA), wherein said RNA sequence has at least 15 nucleotides, wherein said RNA includes a hairpin and wherein said RNA comprises SEQ ID NO.1, SEQ ID NO.2, or a variant of SEQ ID NO.1 or SEQ ID NO.2.

2. Nucleic acid according to claim 1, wherein said hairpin comprises at least 39 nucleotides.

3. Nucleic acid according to claim 1, wherein said RNA sequence has at least 18 nucleotides.

4. Nucleic acid according to claim 1, wherein said RNA sequence has at most 32 nucleotides.

5. Nucleic acid according to claim 1, wherein said target sequence is a part of an exon comprised in said α-syn gene.

6. Nucleic acid according to claim 5, wherein said exon is selected from the group consisting of exon 2, exon 4, and exon 6.

7. Nucleic acid according to claim 5, wherein said part of said exon consists of a sequence selected from the group consisting of SEQ ID NO. 3 to 9 and variants of SEQ ID NO. 3 to 9.

8. Nucleic acid according to claim 1, wherein said RNA sequence comprises one sequence selected from the group consisting of SEQ ID NO. 10 to 16 and variants of SEQ ID NO. 10 to 16.

9. A nucleic acid according to claim 1 wherein the nucleic acid is a DNA molecule.

10.-11. (canceled)

12. An adeno-associated virus (AAV) vehicle comprising a DNA molecule according to claim 9.

13. An AAV vehicle according to claim 12, comprising a capsid comprising an AAV5 or an AAV9 capsid protein sequence.

14. An AAV vehicle according to claim 12, wherein said AAV vehicle is a gene therapy vehicle.

16.-22. (canceled)

23. A method for decreasing and/or knocking down transcripts of encoded by α-syn gene in a subject, wherein the method comprises administering the AAV vehicle according to claim 12 to the subject.

24. The method according to claim 23, wherein the amount of Lewy bodies and/or Papp-Lantos bodies are decreased in the subject.

25. The method according to claim 23, wherein the method is for the treatment or prevention of clinical symptoms of Parkinson's disease (PD), Lewy body Dementia (LBD), multiple system atrophy (MSA), neuropshychiatric symptoms, motor symptoms of PD, cognitive impairment, sleep disturbances, autonomic disturbances, and/or olfactory disturbances.

26. The method according to claim 23, wherein the method is for the treatment or prevention of PD and/or MSA.
